(19) United States
(12) Patent Application Publication
(10) Pub. No.: US 2019/0298681 A1
(43) Pub. Date: Oct. 3, 2019

(54) Nanoliposomal Targeting of Ephrin Receptor A2 (Epha2) and Related Diagnostics

(71) Applicant: Merrimack Pharmaceuticals, Inc., Cambridge, MA (US)

(72) Inventors: Daryl C. Drummond, Lincoln, MA (US); Dmitri B. Kirpotin, San Franscisco, CA (US); Walid Kamoun, Arlington, MA (US)

(21) Appl. No.: 16/085,486

(22) PCT Filed: Mar. 16, 2017
(86) PCT No.: PCT/US2017/022627

§ 371 Date: Sep. 14, 2018

Related U.S. Application Data
(60) Provisional application No. 62/309,215, filed on Mar. 16, 2016.
(60) Provisional application No. 62/322,971, filed on Apr. 15, 2016.

(51) Int. Cl. A61K 31/337; A61K 47/69; A61K 47/68; G01N 33/574; A61P 35/00

(52) U.S. Cl. CPC A61K 31/337; A61K 47/6913; A61K 47/6849; A61P 35/00; G01N 33/57423; G01N 33/57434; G01N 33/57449; G01N 33/57415

(57) ABSTRACT

EphA2 targeted doxorubicin generating nano-liposomes are useful in the treatment of EphA2 positive cancer comprising cancer cells expressing over about 3000 EphA2 receptors/cell. Diagnostic methods for identifying EphA2 positive cancer patients and methods of treating identified patients with a Eph-A2 targeted nanoliposome encapsulating a docetaxel prodrug are provided.

### CROSS-REFERENCE

This patent application claims priority to PCT International National Application No. PCT/US2017/022627, filed Mar. 16, 2017, which claims the benefit under 35 U.S.C. § 119 of U.S. Provisional Application No. 62/322,971 filed on Apr. 15, 2016, and U.S. Provisional Application No. 62/309,215 filed on Mar. 16, 2016. The entire contents of these applications are incorporated herein by reference in their entirety.

### SEQUENCE LISTING

Incorporated by reference in its entirety is a computer-readable sequence listing submitted concurrently herewith and identified as follows: One 48.0 KB ASCII (Text) file named “1107sequence_ST25.txt.”

### TECHNICAL FIELD

This disclosure relates to nano-liposomes targeted to the Ephrin receptor A2, useful in the treatment of EphA2 positive cancer, and related diagnostic methods.

### BACKGROUND

Ephrin receptor A2 (EphA2) is part of the Ephrin family of cell-cell junction proteins highly overexpressed in several solid tumors, and is associated with poor prognosis. The Eph receptors are comprised of a large family of tyrosine kinase receptors divided into two groups (A and B) based upon homology of the N-terminal ligand binding domain. The Eph receptors are involved several key signaling pathways that control cell growth, migration and differentiation. These receptors are unique in that their ligands bind to the surface of neighboring cells. The Eph receptors and their ligands display specific patterns of expression during development. For example the EphA2 receptor is expressed in the nervous system during embryonic development and also on the surface of proliferating epithelial cells in adults. EphA2 also plays an important role in angiogenesis and tumor vascularization, mediated through the ligand ephrin A1. In addition, EphA2 is overexpressed in a variety of human epithelial tumors including breast, colon, ovarian, prostate and pancreatic carcinomas. Expression of EphA2 can also be detected in tumor blood vessels and stromal cells as well.

### SUMMARY

We developed a diagnostic framework for prospective selection of EphA2+ patients for treatment with an EphA-2 targeted nanoliposome encapsulating a docetaxel prodrug, based on a mechanistic single cell cut-off, and a clinical-grade IHC assay. The invention is based in part on the discovery that incubating cells expressing EphA2 with an EphA2-targeted liposome (Example 3) demonstrated specific binding to cells with greater than about 3,000 EphA2 receptors per cell, as determined by the assay methods described herein. For example, FIG. 6 is a graph showing an increase in the liposome-cell association of the EphA2-targeted fluorescent liposomes of Example 3 with cells expressing different levels of EphA2, compared to comparable liposomes that do not include an EphA2 targeting moiety. As used herein, “EphA2 positive” refers to a cancer cell having at least about 3,000 EphA2 receptors per cell (or patient with a tumor comprising such a cancer cell). As shown in FIG. 4, EphA2 positive cells can specifically bind Eph-A2 targeted liposomes per cell. In one example, EphA2 targeted liposomes (e.g., as disclosed in Example 3) can specifically bind to EphA2 positive cancer cells having at least about 3,000 or more (e.g., 75,500 or more) EphA2 receptors. Example 2 describes the IHC assay.

As described in Example 1, we used qFACS and an in vitro assay for liposome (Ls)-cell interaction to identify the minimum number of EphA2 receptors to enable antibody-mediated binding and internalization of Ls. As described in Example 2, we developed an IHC assay able to differentiate EphA2− vs + cell lines. We characterized EphA2 staining pattern in tumor samples of various indications and developed a scoring algorithm that allows selection of patients in early clinical trials.

### BRIEF DESCRIPTION OF THE DRAWINGS

FIG. 1 is a graph showing the liposomal-cell association of 46scFv-ILs and 40scFv-ILs vs. NT-Ls in a panel of cell lines in vitro

FIG. 2 is a graph showing the liposomal-cell association of 46scFv-ILs vs. 40scFv-ILs performed in the same cell lines in vitro

FIGS. 3A, 3B and 3C is a graph showing the expression of EphA2 expressed in receptors per cell for a panel of cell lines quantified using qFACs

FIG. 4 is a graph showing the liposomal-cell association of 46scFv-ILs and 40scFv-ILs and NT-Ls in relation to EphA2 expression in a panel of cell lines in vitro

FIG. 5 is a graph showing the liposomal-cell association of 46scFv-ILs and 40scFv-ILs in relation to EphA2 expression collected in a panel of cell lines in vitro and fitted to a Michaels-Menten equation

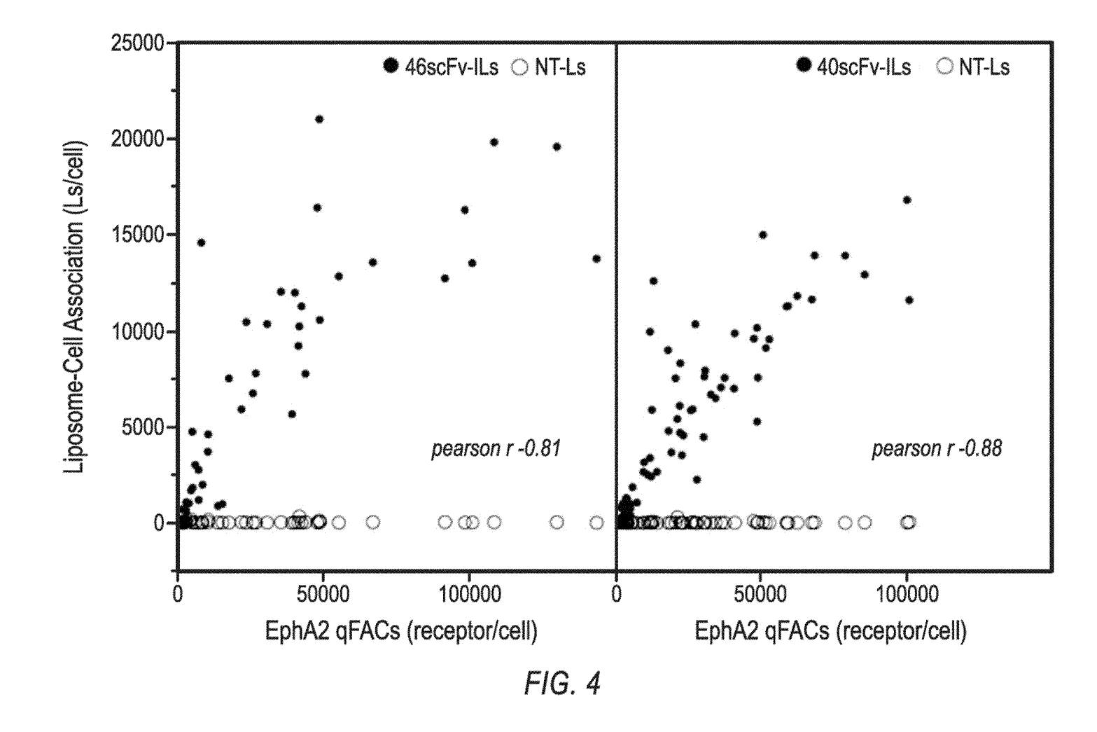

FIG. 6 is a graph in log scale showing the liposomal-cell association of 46scFv-ILs and 40scFv-ILs and NT-Ls in relation to EphA2 expression and identification of cutoffs segregating EphA2 negative (−), EphA2 low (+) and EphA2 high (++) cell lines in vitro

FIG. 7 is a graph showing the Receiver Operating Characteristic Curves for 46scFv-ILs and 40scFv-ILs illustrating the ability of the cutoff of 3000 to correctly classify EphA2 negative from EphA2 positive cells

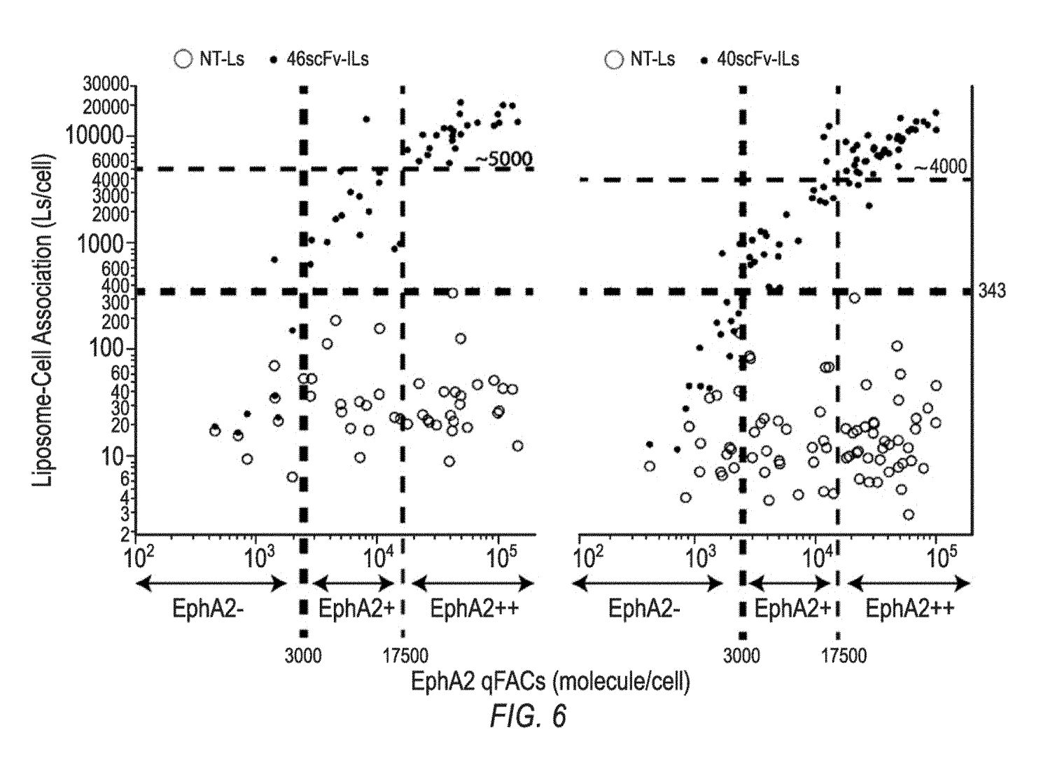

FIG. 8 is a graph showing the brown signal intensity analysis of IHC stained cell arrays at different primary antibody concentrations.

FIG. 9 is a graph showing the correlation between brown signal intensity quantified from IHC stained cell arrays and EphA2 receptor per cells quantified using qFACs

FIG. 10 is an image shows the specificity of the IHC staining illustrated by staining of EphA2 transfected cells vs. EphA2 negative wild type

FIG. 11 is a graph showing the scoring decision matrix for the interpretation of the EphA2 IHC.

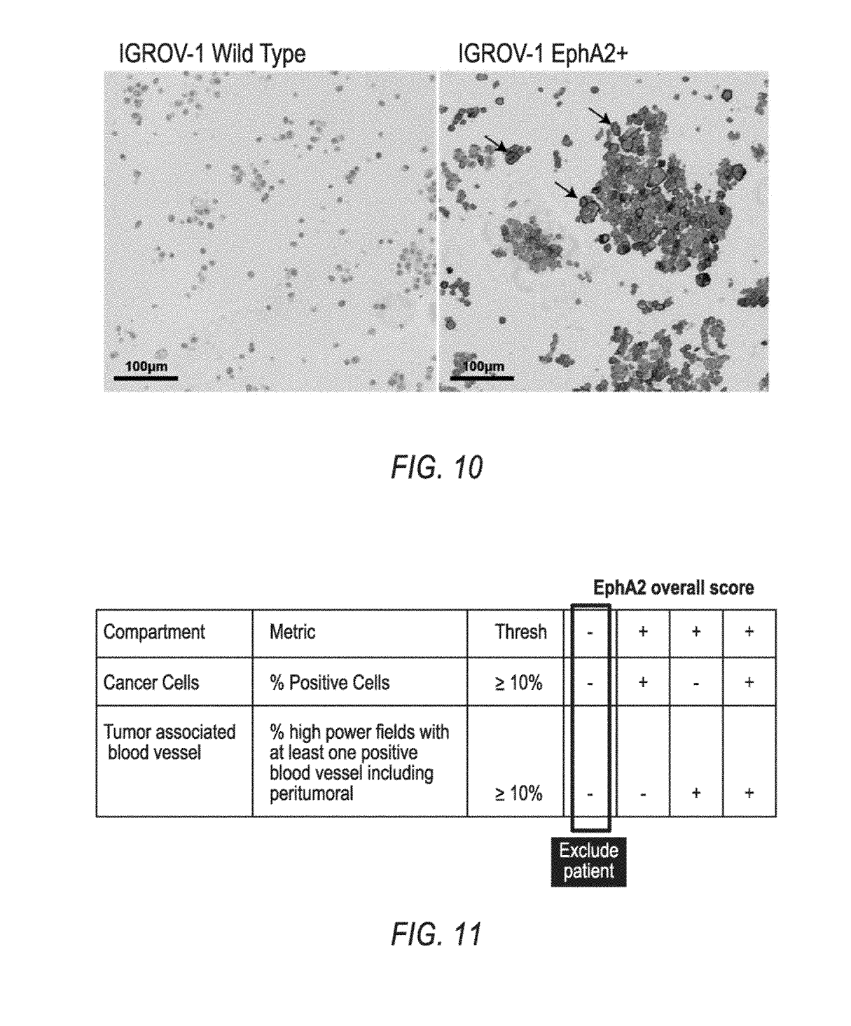

FIG. 12 is a schematic of a docetaxel-generating liposome comprising a EphA2 binding moiety (anti-EphA2 scFv PEG-DSPE).

FIG. 13A is an amino acid sequence and corresponding encoding DNA sequence for the scFv that can be used to prepare EphA2-targeted docetaxel-generating liposomes. The DNA sequence further encodes an N-terminal leader sequence that is cleaved off by mammalian (e.g., human or rodent) cells expressing the encoded scFv.

FIG. 13B is an amino acid sequence and corresponding encoding DNA sequence for the scFv that can be used to prepare EphA2-targeted docetaxel-generating liposomes. The DNA sequence further encodes an N-terminal leader sequence that is cleaved off by mammalian (e.g., human or rodent) cells expressing the encoded scFv.

FIG. 13C is an amino acid sequence and corresponding encoding DNA sequence for the scFv that can be used to prepare EphA2-targeted docetaxel-generating liposomes. The DNA sequence further encodes an N-terminal leader sequence that is cleaved off by mammalian (e.g., human or rodent) cells expressing the encoded scFv.

FIG. 14A is a set of graphs showing EphA2 prevalence and plan for scoring clinical samples.

FIG. 14B shows prevalence of EphA2 in primary tumors and metastases (Ovarian cancer).

### DETAILED DESCRIPTION

EphA2-targeted nanoliposomes can be used to deliver docetaxel (e.g., as an encapsulated docetaxel prodrug) to a cancer cell and/or tumor, leveraging organ specificity through a combination of the enhanced permeability and retention (EPR) effect and cellular specificity through EphA2 targeting. The diagnostic framework disclosed herein can be used, for example, in the clinical implementation of EphA2-based exclusion criteria to select cancer patients to receive an EphA2-targeted nanoliposome containing a docetaxel prodrug, or any other stably associated (T_{1/2} of drug retention greater than 24 h) drug payload.

We developed a novel EphA2-targeted docetaxel nanoliposome, leveraging organ specificity through enhanced permeability effect and cellular specificity through EphA2 targeting. The goal of the study was to develop the diagnostic framework enabling the clinical implementation of EphA2-based exclusion criteria in future trials.

In EphA2 positive tumors (e.g., expressed by either cancer cells or cancer-associate stroma), the EphA2-targeted nanoliposome can bind to EphA2 which can reduce or minimize the washout of liposomes from the tumor, leading to endocytosis of liposomes and the accelerated release of a docetaxel prodrug encapsulated in the EphA2-targeted nanoliposome. Both of these mechanisms are believed to contribute to increased levels of docetaxel delivered to the tumors, both intracellularly and extracellularly, leading to cancer cell death and tumor shrinkage. A key step mediating these mechanisms is the binding of the EphA2-targeted nanoliposome to cells overexpressing EphA2.

“EphA2” refers to Ephrin type-A receptor 2, also referred to as “epithelial cell kinase (ECK),” a receptor tyrosine kinase that can bind and be activated by Ephrin-A ligands. The term “EphA2” can refer to any naturally occurring isoforms of EphA2. The amino acid sequence of human EphA2 is recorded as GenBank Accession No. NP_004422.2.

As used herein, “EphA2 positive” refers to a cancer cell having at least about 3000 EphA2 receptors per cell (or patient with a tumor comprising such a cancer cell). EphA2 positive cells can specifically bind Eph-A2 targeted liposomes per cell. In particular, EphA2 targeted liposomes can specifically bind to EphA2 positive cancer cells having at least about 3000 or more EphA2 receptors per cell.

As used herein, non-targeted liposomes can be designated as “Ls” or “NT-Ls.” Ls (or NT-Ls) can refer to non-targeted liposomes with or without a docetaxel prodrug. “Ls-DTX′” refers to liposomes containing any suitable docetaxel prodrug, including equivalent or alternative embodiments to those docetaxel prodrugs disclosed herein. “NT-Ls-DTX” refers to liposomes without a targeting moiety that encapsulate any suitable docetaxel prodrug, including equivalent or alternative embodiments to those docetaxel prodrugs disclosed herein. Examples of non-targeted liposomes including a particular docetaxel prodrug can be specified in the format “Ls-DTXp[y]” or “NT-DTXp[y]” where [y] refers to a particular compound number specified herein. For example, unless otherwise indicated, Ls-DTXp1 is a liposome containing the docetaxel prodrug of compound 1 herein, without an antibody targeting moiety.

As used herein, targeted immunoliposomes can be designated as “ILs.” Recitation of “ILs-DTXp” refers to any embodiments or variations of the targeted docetaxel-generating immunoliposomes comprising a targeting moiety, such as a scFv. The ILs disclosed herein refer to immunoliposomes comprising a moiety for binding a biological epitope, such as an epitope-binding scFv portion of the immunoliposome. Unless otherwise indicated, ILs recited herein refer to EphA2 binding immunoliposomes (alternatively referred to as “EphA2-ILs”). The term “EphA2-ILs” refers herein to immunoliposomes enabled by the present disclosure with a moiety targeted to bind to EphA2. ILs include EphA2-ILs having a moiety that binds to EphA2 (e.g., using any scFv sequences that bind EphA2). Preferred targeted docetaxel-generating immunoliposomes include ILs-DTXp3, ILs-DTXp4, and ILs-DTXp6. Absent indication to the contrary, these include immunoliposomes with an EphA2 binding moiety and encapsulating docetaxel prodrugs of compound 3, compound 4 or compound 6 (respectively). EphA2-ILs can refer to and include immunoliposomes with or without a docetaxel prodrug (e.g., immunoliposomes encapsulating a trapping agent such as sucrose octasulfate without a docetaxel prodrug).

The abbreviation format “[x]scFv-ILs-DTXp[y]” is used herein to describe examples of immune-liposomes (“ILs”) that include a scFv “targeting” moiety having the amino acid sequence specified in a particular SEQ ID NO:[x], attached to a liposome encapsulating or otherwise containing a docetaxel prodrug (“DTXp”) having a particular Compound number ([y]) specified herein. Unless otherwise indicated, the scFv sequences for targeted ILs can bind to the EphA2 target.

The term “NT-Ls” refers to non-targeted liposomes enabled by this disclosure without a targeting moiety. The term “NT-Ls-DTX′” refers to a non-targeted liposomes enabled by this disclosure encapsulating a docetaxel prodrug (“DTX′”).

The minimum EphA2 expression required for sufficient binding of the liposome was analyzed (Example 1) to determine the relationship between EphA2 expression (measured by qFACs) and target-mediated liposome-cell association in vitro using a panel of cell lines.

As described in Example 1, EphA2 targeted liposome/cell interaction directly correlated with expression target, while non-targeted liposome interaction with cells was minimal and not affected by target expression. The cutoff that can stratify cell lines based on EphA2-ILs/cell interaction was determined by assessing non targeted liposome/cell association and established as the value for the highest non targeted liposome/cell association (343 liposomes/cell). We next used a statistical partition method to determine the optimal EphA2 expression cutoff (≈3,000 receptors/cell) with minimal misclassification

While non targeted Ls do not associate with cells in vitro, there is a strong correlation between EphA2 expression and EphA2-ILs cell association independent of the cell line origin. We used the non-targeted Ls to determine the extent of non-specific binding that can be achieved (^{˜}340 Ls/cell) and used partitioning to determine the minimum number of EphA2 receptors necessary to mediate targeting (^{˜}3,000 receptors/cell). We have developed and validated a qIHC assay for EphA2 (precision ^{˜}90%, linearity 0.8 and reproducibility ^{˜}5%). We stained a set of ^{˜}200 tumor samples from various indications. EphA2 was found to be expressed in tumor cells, tumor-associated myofibroblasts, and tumor-associated blood vessels. Using an inclusive cutoff of 10%, EphA2 prevalence was found to range from 50% to 100% in the tumor types evaluated. No significant difference in staining was seen between metastasis and primary tumors in matched samples.

Results are summarized in Table 1 below.

TABLE 1

| | | Tumor | Tumor | |
|---|---|---|---|---|
| | | associated | associated | EphA2 Overall |
| | Cancer Cells | myofibroblasts | blood vessels | Score |
| Bladder | 19/20 (95%) | 0/20 (0%) | 16/20 (80%) | 19/20 (95%) |
| Gastric | 18/20 (90%) | 3/20 (15%) | 17/20 (85%) | 20/20 (100%) |
| Head & | 16/19 (84%) | 0/19 (0%) | 9/19 (47%) | 19/19 (100%) |
| Neck | | | | |
| Lung | 24/41 (58%) | 1/41 (2.4%) | 24/41 (58%) | 28/41 (68%) |
| Ovarian | 10/18 (55%) | 7/18 (39%) | 17/18 (95%) | 17/18 (95%) |
| Pancreatic | 15/19 (79%) | 0/19 (0%) | 11/19 (58%) | 17/19 (89%) |
| Prostate | 7/23 (27%) | 7/23 (27%) | 9/23 (28%) | 12/23 (52%) |
| TNBC | 6/77 (7%) | 0/77 (0%) | 34/77 (44%) | 37/77 (48%) |

### EXAMPLES

### Example 1: Analysis of Cell Expression of the Target Effect on Liposome Targeting In Vitro

Example 1 details the characterization of an exemplary Eph-A2 targeted Liposome of Example 3 (herein “EphA2-ILs”), with respect to its ability to bind to tumor cells and establishes a cutoff value of EphA2 expression that is sufficient for EphA2-ILs binding. By comparing results from a screening assay assessing the binding affinity of immunoliposomes incorporating two EphA2 targeting clones 40scFv and 46scFv with a non-targeted liposome (NT-Ls), we have established that there is a high correlation in binding capacity between clones (R2=0.97). Furthermore, immunoliposomes with both EphA2 clones exhibited a statistically significant increase in binding (P<0.0001) compared to an untargeted liposomal control. Subsequent analysis determined that, of the two clones tested, 46scFv-ILs exhibited a higher 49% increase in liposome/cell association than clone 40scFv. In addition, qFACS analysis used to quantify EphA2 expression showed high level of specificity of EphA2 targeted liposomes to EphA2 positive cells. There was a strong correlation (pearson correlation>0.8) between EphA2 targeted liposome association with cells and EphA2 expression.

Materials

TABLE 2
Reagents:

| Reagent | Source | Cat# |
|---|---|---|
| Quantibrite Beads | BD Bioscience | Cat # 34095 |
| EphA2 PE Ab | R&D | Cat # FAB3035P |
| FACScalibur | BD Bioscience | |

Quantibrite beads from BD were used to create a standard curve for number of PE (phycoerythrin) molecules per beads. Following Becton Dickinson's instructions, for each experiment 500 ul of FACs buffer was added to the supplied tube and subsequently read on a BD FACs Calibur flow cytometer previously calibrated with Right Reference beads.

Cells were cultured in the appropriate media (see cell line char) until ^{˜}70-80% confluent then trypsinized, counted, and washed in FACs buffer to obtain a final concentration of 4×10\hat{ }6 cells/well in each well a 96 well round bottom plate. Cells were then incubated with 200 nM of R&D system's EphA2 PE antibody for 20 minutes on ice, washed and resuspended in 100 ul of FACs buffer. The cells were read on the BD FACs Calibur flow cytometer and data was expressed as described with respect to the qFACs method validation herein.

Liposome-Cell Association Assay: Cell Uptake of Covalently scFv-Conjugated Liposomes

Liposomes are prepared by ethanol injection-extrusion method. For sphingomyelin (SM) liposomes, lipids are comprised of sphingomyelin, cholesterol and PEG-DSG (3:2:0.24 molar parts), with either DiIC18(3)-DS (DiI3-Ls), or DiIC18(5)-DS (DiI5-Ls) fluorescent lipid labels added at a ratio of 0.3 mol % of the total phospholipid. Briefly, for a 30 ml liposome preparation, lipids are dissolved in 3 ml ethanol in a 50-ml round bottom flask at 70 Celsius. HEPES-buffered saline (5 mM HEPES, 144 mM NaCl, pH 6.5) is warmed at 70 Celsius water bath to above 65 Celsius and mixed with the lipid solution under vigorous stirring to give a suspension having 50-100 mM phopsholipid. The obtained milky mixture is then repeatedly extruded, e.g., using thermobarrel Lipex extruder (Northern Lipids, Canada) through 0.2 μm and 0.1 μm polycarbonate membranes at 65-70° C. Phospholipid concentration is measured by phosphate assay. Particle diameter is analyzed by dynamic light scattering. Liposomes prepared by this method have sizes about 95^{˜}115 nm. Anti-EphA2 scFv proteins were expressed in mammalian cell culture, purified by protein A affinity chromatography, and conjugated through C-terminal cysteine residue to maleimide-terminated lipopolymer, mal-PEG-DSPE, in aqueous solution at 1:4 protein/mal-PEG-DSPE molar ratio. The resulting micellar scFv-PEG-DSPE conjugates were purified by gel chromatography on Ultrogel AcA34 or AcA44 (Sigma, USA). Anti-EphA2 scFv proteins were expressed in mammalian cell culture, purified by protein A affinity chromatography, and conjugated through C-terminal cysteine residue to maleimide-terminated lipopolymer, mal-PEG-DSPE, in aqueous solution at 1:4 protein/mal-PEG-DSPE molar ratio. The resulting micellar scFv-PEG-DSPE conjugates were purified by gel chromatography on Ultrogel AcA34 (Sigma, USA). Targeted DiI3-Ls or DiI5-Ls were prepared by incubation with micellar anti-EphA2 scFv-PEG-DSPE conjugate at 60° C. for 30 min at the scFv/liposome ratio of 10-12 g/mol phospholipid for 40scFv-ILs, and 5 g/mol phospholipid for 46scFv-ILs. The ligand inserted liposomes are purified on Sepharose CL-4B column and analyzed by phosphate assay for lipid concentration and SDS-PAGE for antibody quantification.

Cells used in this study should be at 70-90% confluence. 24 hours prior to the study, media was replaced with a fresh aliquot of RPMI (containing 10% FBS, 2 mM glutamine and pep/strep) and harvested by trypsinization. The cells were then resuspended in growth medium, plated out at 100,000 cells per well, washed and incubated with 100 ul of media containing 50 μM phospholipid liposomes. Subsequently, the cells were incubated in the dark at 37° C. for 4 hours with constant shaking. After that time the cells were washed 2-3 times with PBS and resuspended in 100 ul/well PBS for the FACS analysis. The mean cell fluorescence (MCF) of the DiI5 labeled liposomes was determined using FACScalibur (BD bioscience). The observed fluorescence signal is representative of both surface-bound and internalized nanoparticles while the MCF of the cells incubated with blank liposomes (no conjugated scFv) was used to determine non-specific bindings.

TABLE 3
Cell lines:

| Cell Line | Tumor Type | Media |
|---|---|---|
| ADRr | Ovarian | RPMI 10% FBS |
| | | 1% pen/strep |
| BT-474-SO | Breast | RPMI 10% FBS |
| | | 1% pen/strep |
| BT-549 | Breast | RPMI 10% FBS |
| | | 1% pen/strep |
| ES-2 | Ovarian | McCoy's 5A 10% FBS |
| | | 1% pen/strep |
| HCC 1806 | Breast | RPMI 10% FBS |
| | | 1% pen/strep |
| HCC 1954 | Breast | RPMI 10% FBS |
| | | 1% pen/strep |
| IGROV-1-SO | Ovarian | RPMI 10% FBS |
| | | 1% pen/strep |
| IGROV-1-TK | Ovarian | RPMI 10% FBS |
| | | 1% pen/strep |
| MDA-MB-231 | Breast | RPMI 10% FBS |
| | | 1% pen/strep |
| MDA-MB-436 | Breast | L-15 10% FBS |
| | | 1% pen/strep |
| MDA-MB-468 | Breast | L-15 10% FBS |
| | | 1% pen/strep |
| OV-90 | Ovarian | RPMI 10% FBS |
| | | 1% pen/strep |
| OVCA 433 | Ovarian | RPMI 10% FBS |
| | | 1% pen/strep |
| SUM-149 PT | Breast | DMEM/F12 10% FBS- |
| | | 1% pen/strep- |
| | | Hydrocortisone/insulin |
| SUM-159 PT | Breast | DMEM/F12 10% FBS- |
| | | 1% pen/strep- |
| | | Hydrocortisone/insulin |
| SW626 | Ovarian | RPMI 10% FBS |
| | | 1% pen/strep |
| BT 474-TK | Breast | RPMI 10% FBS |
| | | 1% pen/strep |
| BT-20 | Breast | RPMI 10% FBS |
| | | 1% pen/strep |
| COV 362 | Ovarian | RPMI 10% FBS |
| | | 1% pen/strep |
| COV 504 | Ovarian | RPMI 10% FBS |
| | | 1% pen/strep |
| HCC 1937 | Breast | RPMI 10% FBS |
| | | 1% pen/strep |
| HCC 38 | Breast | RPMI 10% FBS |
| | | 1% pen/strep |
| HCC 70 | Breast | RPMI 10% FBS |
| | | 1% pen/strep |
| Hs 578T | Breast | DMEM 10% FBS |
| | | 1% pen/strep |
| MCF-7 | Breast | RPMI 10% FBS |
| | | 1% pen/strep |
| MDA-MB-453 | Breast | L-15 10% FBS |
| | | 1% pen/strep |
| OV 17R | Ovarian | RPMI 10% FBS |
| | | 1% pen/strep |
| OVCAR 5 | Ovarian | RPMI 10% FBS |
| | | 1% pen/strep |
| OVCAR-8 | Ovarian | RPMI 10% FBS |
| | | 1% pen/strep |
| SKBr-3 | Breast | McCoy's 5A 10% FBS |
| | | 1% pen/strep |
| SKOV-3 | Ovarian | RPMI 10% FBS |
| | | 1% pen/strep |
| TOV-112D | Ovarian | RPMI 10% FBS |
| | | 1% pen/strep |
| CaOV 3 | Ovarian | DMEM 10% FBS |
| | | 1% pen/strep |
| COV 318 | Ovarian | RPMI 10% FBS |
| | | 1% pen/strep |
| MDA-MB-157 | Breast | L-15 10% FBS |
| | | 1% pen/strep |
| OVCAR 3 | Ovarian | RPMI 10% FBS |
| | | 1% pen/strep |
| OVCAR 4 | Ovarian | RPMI 10% FBS |
| | | 1% pen/strep |
| SUM 190 | Breast | DMEM/F12 10% FBS- |
| | | 1% pen/strep- |
| | | Hydrocortisone/insulin |
| COV 434 | Ovarian | RPMI 10% FBS |
| | | 1% pen/strep |
| HCC 1187 | Breast | RPMI 10% FBS |
| | | 1% pen/strep |
| OV 56 | Ovarian | RPMI 10% FBS |
| | | 1% pen/strep |
| OV 7 | Ovarian | RPMI 10% FBS |
| | | 1% pen/strep |
| NCI-H187 | Lung | RPMI 10% FBS |
| | | 1% pen/strep |
| KLN205 | Mouse Lung | EMEM 10% FBS |
| | | 1% pen/strep |
| SHP-77 | Lung | RPMI 10% FBS |
| | | 1% pen/strep |
| NCI-H345 | Lung | RPMI 10% FBS |
| | | 1% pen/strep |

Assay Validation

This assay aims to assess target-mediated liposome-cell association in order to quantify the uptake of covalently scFv-conjugated liposomes vs non targeted liposome. We have tested two clones of the EphA2 antibody 46scFv-ILs and 40scFv-ILs. Briefly, cells are incubated with either targeted or non-targeted liposomes fluorescently labeled with a lipophilic fluorophore for 4 hours then washed and measured for single cell fluorescence using flow cytometry. Fluorescent beads with a known number of fluorophores were used as standard curve to determine the number of liposomes from mean fluorescence values. Overall, the assay demonstrated high linearity (mean R²=0.98), reproducibility (intercept and slope for the standard curve within 10%) and low intra-assay variability (average CV between technical replicates=2.1% [0.03%-19%]). A subset of cell lines (20%=13/65) was run twice and data shows reproducible Liposomal uptake between runs: RUN#1 labeled as POC10 and RUN#2 labeled as RUN#74. For assay reproducibility 40scFv-ILs was used. In order to back calculate number of liposomes and an estimated docetaxel load, we use the following equations.

$$PhL = \frac{{n}_{Cy 5}}{{f}_{DiI 5}} * {10}^{6} * \frac{L}{a}$$

### PhL: Amount of Liposomal

Phospholipids in nmoles/million cells
n_{cys}: number of fluorophores calculated from beads standard curve
f_{Dil5}: molar percentage of Dil5 per liposome (=0.216)
L: Avogadro number in nmoles (6.02×10¹⁴)
a: Quantum yield correction between the beads Cy5 and the liposomal Dil5(=3.76±0.25 measured)

$${n}_{Ls} = \frac{{PhL}_{EphA 2} - {PhL}_{nT}}{{Ls}_{PhL} * 10 ⋀ 6} * L$$

n_{Ls}: number of Liposomes associated to a cell due to EphA2 targeting
PhL_{EphA2}: amount of EphA2 targeted liposomes
PhL_{nT}: amount of non-targeted liposomes
L_{SPHL}: number of phospholipid molecules per liposome (=80 10⁴)
L: Avogadro number in nmoles (6.02×10¹⁴)

$${n}_{Docetaxel} = \frac{{PhL}_{EphA 2} - {PhL}_{nT}}{{Docetaxel}_{load}}$$

n_{Docetaxel}: Predicted amount of Docetaxel delivered due to EphA2 targeting in ngram/million cells
PhL_{EPhA2}: amount of EphA2 targeted liposomes
Ph_{LnT}: amount of non-targeted liposomes
Docetaxel_{load}: amount of docetaxel loaded per liposome in gram of docetaxel/mole of PhL

$${C}_{Docetaxel} = \frac{{n}_{Docetaxel} * {10}^{3}}{V}$$

C_{Dweiaxel}: Predicted concentration of Docetaxel compatible with in vitro IC50experiments in ng/ml
V: volume of incubation media in 384 well plate (50 ul)

To validate the liposome-cell association assay, we tested the reproducibility in two biological replicates. POC10 and Run#74 represent two runs of the assay performed one month apart and done over 4 days for each run. No significant difference between the two runs for EphA2-Liposome (40scFv-ILs) and NT-Liposome levels. B. Standard curves from Run#74 performed with every flow cytometric run and shows linearity and stability of the assay.

TABLE 4
Liposome-cell association reproducibility & linearity

| | Intercept | Slope | R² |
|---|---|---|---|
| Test 1 | 4.06 | 7.06E−04 | 0.99990 |
| Test 2 | 3.17 | 6.35E−04 | 0.99993 |
| Test 3 | 2.25 | 7.11E−04 | 0.99995 |
| Test 4 | 1.25 | 7.07E−04 | 0.99999 |

EphA2-Targeted Vs. Non Targeted Liposome-Cell Association

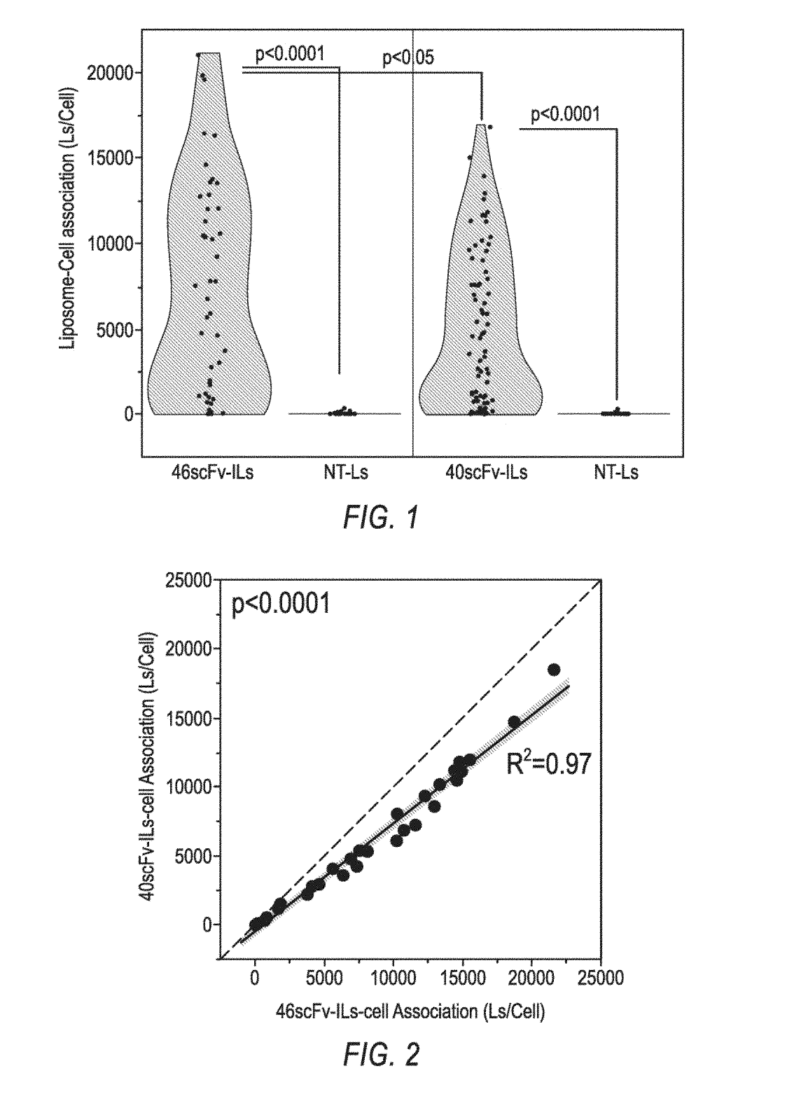

FIG. 1 shows the results of an analysis conducted to characterize the range of liposome-cell association. We screened a large set of cell lines using either 40scFv-ILs or 46scFv-ILs and compared it to NT-Ls. We found that EphA2-ILs associated with cells, in a statistically significant manner, more than NT-Ls independent of the EphA2 clone used. Referring to FIG. 1 (EphA2-ILs vs NT-Ls cell association), a set of cell lines was tested with EphA2 targeted immunoliposomes 46scFv-ILs and 40scFv-ILs. EphA2-ILs demonstrated statistically significantly high association with cells when compared to NT-Ls (paired t-test). Both EphA2 antibody clones had a similar binding, with a small but statistically significant higher level of association with 46scFv-ILs.

To determine the correlation between clones, a set of 34 cell lines were assessed side by side. Both clones showed a high correlation to each other as indicated by an R2 value of 0.97. However, 46scFv-ILs led to a statistically significant higher liposome-cell association than clone 40scFv-ILs (p<0.0001), with on average 49% increase in the number of liposomes per cell and a standard deviation of 21%.

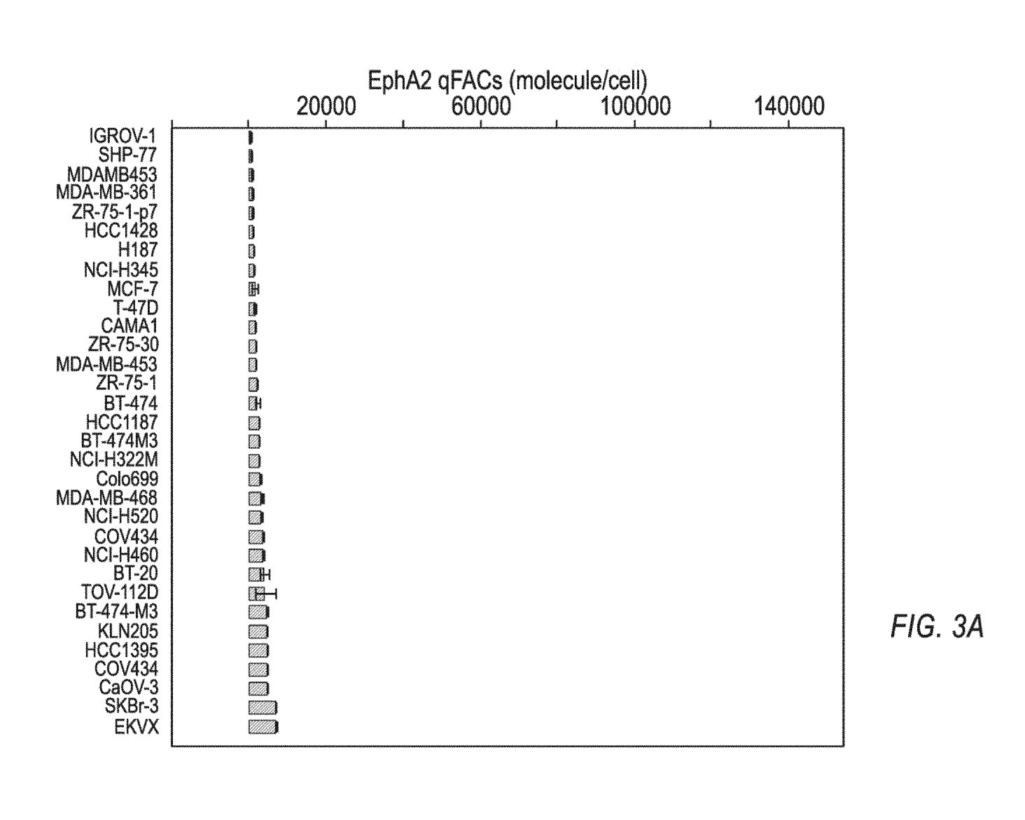

FIG. 2 (40scFv-ILsys 46scFv-ILs Ls cell association) shows the results of an analysis of 34 cell lines used to compare the two EphA2 scFv clones in the same study. A strong linear correlation is seen between both antibody clones, with a significantly higher liposome-cell association for 46scFv-ILs.

EphA2 qFACs Assay Validation

The EphA2 qFACS assay aims to quantify EphA2 molecules per cell using quantitative flow cytometry (qFACs). To summarize, cells are incubated with EphA2 antibody (R&D Clone 3035 mouse monoclonal) conjugated to PE for 1 hour. The cells of interest are then washed and assessed for fluorescence intensity using flow cytometry. PE labeled beads (Quantibtrite™ PE-quantitation kit, BD bioscience) are concomitantly analyzed using flow cytometry and subjected to linear regression analysis to back calculate the number of antibodies bound to each cell. We assume that one antibody can only bind to one antigen, thus the number of antibodies is equal to the number of receptors per cell. In terms of assay performance, the assay is highly linear (mean R²=0.99) and reproducible (intercept and slope for the standard curve within 10%) and theintra-assay variability was low (average CV between technical replicates=5.6% [0.6%-37%]). A subset of cell lines was run twice and data shows reproducible EphA2 levels. Referring to theEphA2 qFACs assay validation, two runs of the assay were performed one month apart and done over 4 days for each run. No significant difference between the two runs for EphA2 levels. In addition, standard curves were performed with every qFACs run and shows linearity and stability of the assay.

Characterization of EphA2 Expression in Cancer Cell Lines

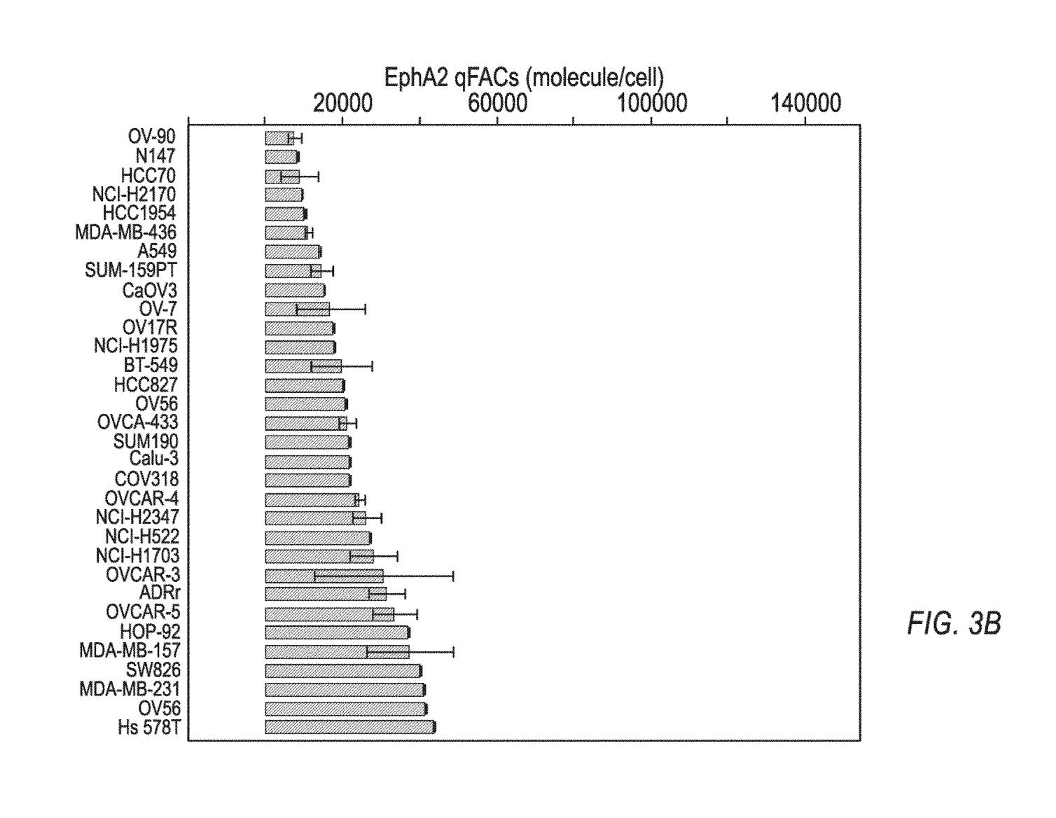

We performed qFACs and liposome-cell association studies on the same day and using the same batch of cells. qFACs data shows that EphA2 ranged from 422 to 143,888 receptors per cell (FIG. 3A, 3B, 3C).

EphA2 Expression Vs. Target-Mediated Liposome-Cell Association

The goal of this analysis is to assess the correlation between EphA2 expression and target mediated liposome-cell association. We found a significant correlation between EphA2 expression and EphA2-ILs-cell association (Pearson correlation coefficient=0.81 for 46scFv-ILs and 0.88 for F5-10A7) which was independent of the EphA2 antibody clone.

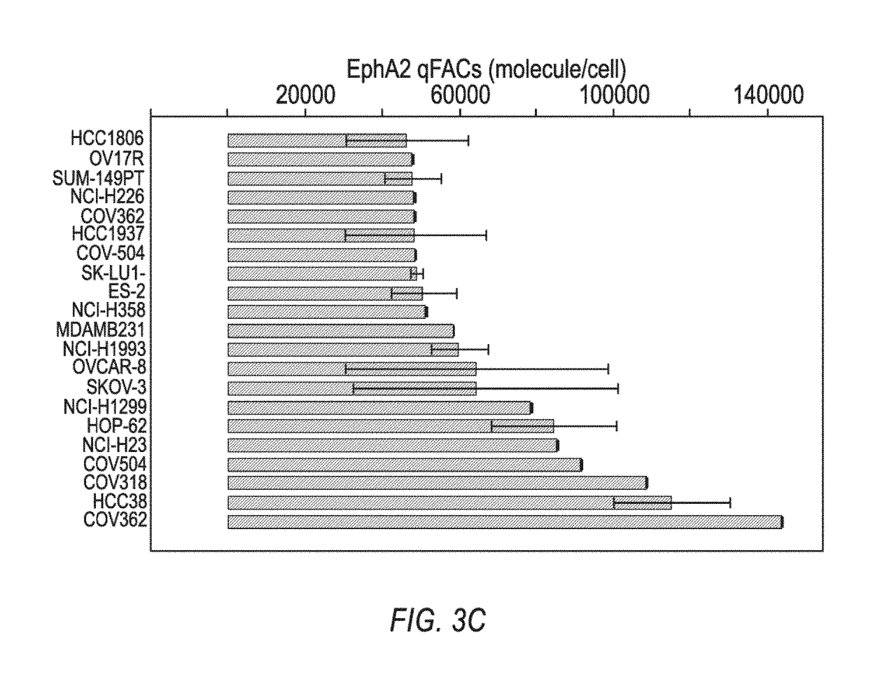

Referring to FIG. 4 (EphA2 expression in a panel of cancer cell lines), the relationship between EphA2 expression and target-mediated liposome association is best fitted by a Michaelis-Menten equation suggesting standard antigen-antibody binding kinetics and was independent of cell line origin.

$$Michaelis \text{-} Menten \frac{( a * Qfacs average )}{( b + Qfacs average )} \begin{matrix}AIC = Max Reaction Rate \\ BIC = Inverse Affinity\end{matrix}$$

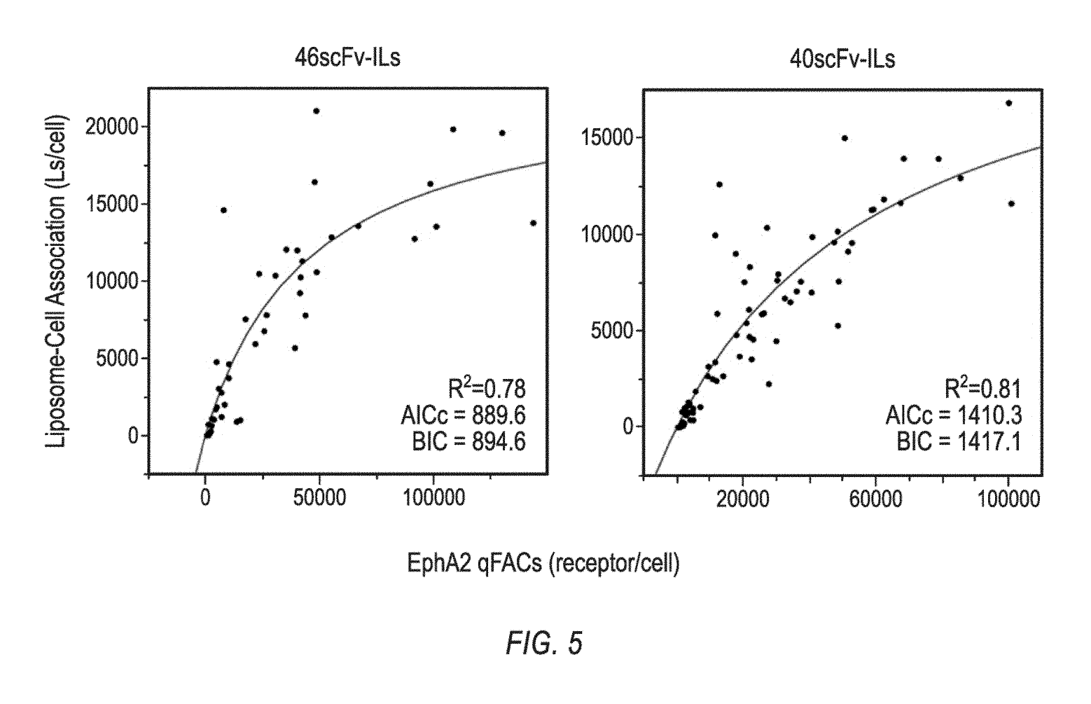

Given the relationship between EphA2 expression and target-mediated liposome-cell association, we have identified a cutoff that can classify the cell lines. The cutoff was determined by assessing non-targeted liposome-cell association and established by taking the 99 percentile of NT-Ls-cell association which is about 340 liposomes/cell. We used a statistical partition method to determine the optimal EphA2 expression cutoff (about 3000 receptors/cell) with minimal misclassification (=1% error). This cutoff separates targeting-negative from targeting-positive cell lines. FIG. 6 is a graph showing EphA2 expression in a panel of specified selected cancer cell lines.

While the first cutoff is derived from non-targeted liposome-cell association, the second cutoff (separates EphA2+ from EphA2++ FIG. 6), was determined by looking for clustering spots in the data segregating large number of cells. The identified clustering spot was performed in 46scFv-ILs and then extrapolated to 40scFv-ILs. We identified 5,000 liposome/cell as the next level which through partition analysis leads to about 17,500 receptors/cell. Given that 40scFv-ILs has lower liposome-cell association than 46scFv-ILs, the 17,500 receptors/cell correlated with about 4,000 liposomes/cell. For both partition analysis, the error of partition calculated as area under the curve of ROC was 0.94 and 0.98 for 40scFv-ILs and 46scFv-ILs respectively.

### Example 2: Quantitative Assessment of IHC Assay Performance

This example describes the EphA2 IHC CDx assay. The assay was tuned to allow visual detection of EphA2 expression matching the identified cutoff of 3000 receptors/cell. The assay demonstrated acceptable levels of sensitivity, specificity and precision. All the planned tasks were completed and the EphA2 IHC CDx demonstrated specificity and sensitivity for EphA2 staining and had solid precision as defined by using quantitative image analysis.

The EphA2 IHC CDx showed high level of specificity and sensitivity when tested in a set of cancer cell lines with a range of EphA2 expression. Intra-assay and inter-assay variability was very low in cell lines and tissue samples.

Materials & Methods

Cell array and TMA maps are found in Appendix A, and described in Table 5. All tissue samples were selected to include all the relevant tumor types that will be included in the Phase 1 trial. For all the cell lines we focused on the three tumor types from which we included a large set of cell panel.

TABLE 5
List of TMAs used for assay qualification

| | | Intended |
|---|---|---|
| Sample ID | Description | Analysis |
| Human | 24 tumor samples: 3 samples × 8 tumor | Precision |
| Tissue Micro | types. | |
| Array: | Sample QC: | |
| HTMA060915 | <=50% missing cores | |
| | Cores must be identifiable-non | |
| | identifiable cores are excluded | |
| | Indications: Gastric, Bladder, Lung, Breast, | |
| | Prostate, Pancreatic, Ovarian, Head & | |
| | Neck | |
| Cell Array: | 78 unique samples collected from 65 cell | Precision |
| CA022515 | lines with known EphA2 levels. | Sensitivity |
| | Sample QC: | Specificity |
| | <=30% missing cores | |
| | Cores must be identifiable-non | |
| | identifiable cores are excluded | |
| | Indications: Lung, Ovarian, Breast | |
| Cell Array: | Duplicates of EphA2 transfected and | Specificity |
| CA111014 | parental cell lines. | |
| | Sample QC: | |
| | At least one pair of cell lines | |
| | Indication: Ovarian | |

EphA2 IHC CDx Sectioning and Staining Protocol Performed on Dako Autostainer Instrument

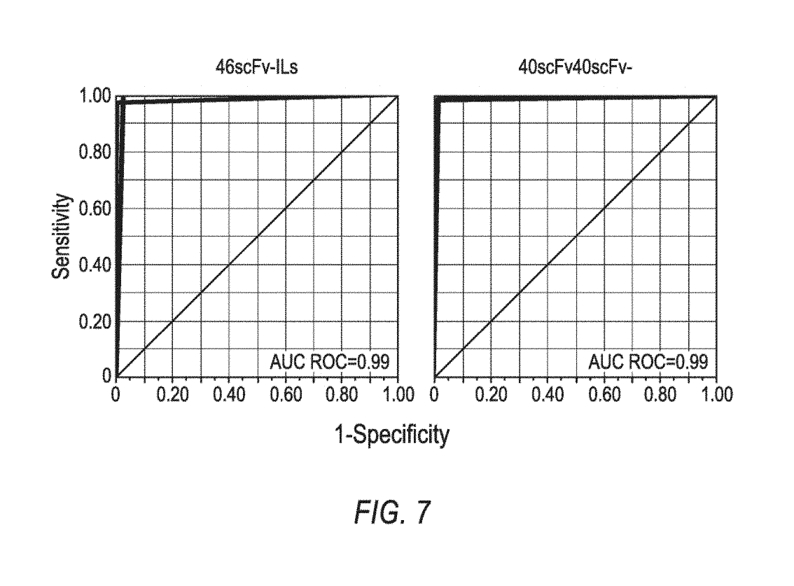

Tissue sections were cut at 5 micron thickness and mounted on positively charged slides for immunohistochemistry analysis. Primary antibody used: Rabbit mAb EphA2 (D4A2) (Cell Signaling Technology-22050BF) used at 1:1000 dilution to a working concentration of 2 μg/ml. A range of concentrations were tested and acceptable concentration was identified as low as 1 μg/ml and up to 10 μg/ml (FIG. 8).

Labelled Polymer Used: En Vision+ System-HRP Labelled Polymer Anti-Rabbit (DAKO K400311)

1. General procedure

2. + Deparaffinization

3. + Antigen Retrieval: 25 minutes @ 102° C.

4. + Endogenous Enzyme Block (Peroxidased): 10 minutes

5. + Buffer Rinse: 4 minutes

6. + Protein Block: 10 minutes

7. + Buffer Rinse: 4 minutes

8. + Primary Antibody at a dose of 2 μg/nnl for 60 minutes

9. + Buffer Rinse: 4 minutes

10. + Labelled Polymer: 30 minutes

11. + Buffer Rinse: 4 minutes

12. + Flex DAB+ Substrate-Chromogen: 10 minutes

13. + Buffer Rinse: 4 minutes

14. + Auto Hematoxylin: 6 minutes

15. + Buffer Rinse: 4 minutes

16. + Coverslip

Quantitative Flow Cytometry

Cell lines characterized in previous example (example 1) were used to evaluate the performance of the assay. In summary, qFACs was used to quantify EphA2 receptor per cell in 65 cell lines (13 of which were done in duplicates). EphA2 expression ranges from 422 to 143,888 receptors per cell.

Formalin Fixed Paraffin Embedded (FFPE) Cell Pellets

Each cell line was processed to expanded and processed to mimic clinical samples leading to the generation of formalin fixed paraffin embedded cell pellets. In summary, cells were expanded to 50-200 million cells, washed with PBS, tripsinzed using 0.05% trypsine, centrifuged and washed in PBS, fixed in 10% formalin for 2-4 hours prior to switching them to 70% ethanol. Cells were stored at 4° C. in 70% ethanol for up to one week. Cells embedded in histogel at a density of 1×10⁵/μl of histogel. Histogel embedded cell pellets are stored in 70% ethanol prior to standard processing in paraffin embedding processor. From FFPE blocks, cell arrays were generated by extracting 2 mm cores from each block and transferring them to a cell array block.

Cell Line Transfection

In order to generate EphA2 overexpressing cell lines we used ready to go particle (GeneCopoeia, Rockville, Md.). The construct is based on pReceiver-Lv105, a Puromycin selectable lentiviral vector.

EphA2 Gene Info:

NM_004431.1. Virus cat#: LP-A0125-LV105-0205. The info and protocol can be downloaded at www.sigmaaldrich.com/content/dam/sigma-aldrich/docs/Signna/General_Infornnation/lentiviraltransdprotocol.pdf.

However, we adjusted and modified the protocol of infection as needed. Our protocol for the IGROV-1 cell lines was performed as following: Day1, count and seed cells into a 96-well (4,000 cells per well in 100 ul media) plate; Day2, 1) remove media and add 100 μl infection solution containing Polybrene [e.c. 8 μg/ml] and 5 μl [low MOI] or 30 ul [high MOI] EphA2 expressing virus. 2a) spin at room temperature for 90 min at 2300 rpm and leave overnight in incubator at 37° C. 2b) if cells show Polybrene sensitivity, add the same day 150 μl/well fresh growth media after 6 h of incubation. 2c) alternatively, reduce Polybrene final concentration down to 4 μg/ml. 2d) if cells show sensitivity towards the long spin, reduce time to 30 min and increase temperature to 30° C.; day 3, remove all media and replace with 200 μl fresh growth media, day 4 rest, day 5 start 5 days Puromycin selection by replacing the media with 2 μg/ml Puro containing growth media, and day 6 test EphA2 expression levels by FACS or similar methods.

Validation Approach

To assess intra-assay and inter-assay precision slides were stained together in triplicates three times generating stained slides (Run1.1, Run1.2, Run1.3, Run2.1, Run2.2, Run2.3, Run3.1, Run3.2, Run3.3). Staining runs were performed on different days (Day 1, Day 3, Day 5) with two different operators. 1) Day 1—Note: The assay run for the intra-assay precision represents the samples on Day 1 of the inter-assay precision as well. 2) Day 3—The assays will be repeated on a second replicate set of 3 unstained slides from the same TMA blocks used on Day 1. 3) Day 5—The assays were repeated on a third replicate set of 3 unstained slides from the same TMA blocks used on Day 1 and Day3

Microscopy and Image Analysis

Images were collected with an AperioBF Scanscope (Leica Biosystems, Buffalo Grove Ill.) at 20× magnification. Quantitation of EphA2 brown signal was done using an in-house algorithm and user interface developed using Matlab (Mathworks, Natick, Mass.). In summary, cells or tissue areas were segmented using a semi-automated threshold based algorithm in which the user can change the threshold or manually include or exclude areas. A core annotation tool is used to match the cores to the sample ID, which is QCed by the user for every image. Snapshot images are stored for further QC. A higher resolution tissue segmentation algorithm is used to tighten the mask around the tissue or cells. From each core, an average brown stain intensity is computed by converting the RGB image to the color space CYMK and using the yellow channel as the best representative for the brown color. Mean brown signal intensity will be captured for each cell line or tumor within the TMA and used for sensitivity, specificity, and precision calculations.

Statistical Analysis

All statistical analyses were performed using JMP (SAS, NC). For analysis of the cell lines, CA022515, linear regression was performed to assess the linearity of EphA2 IHC brown intensity vs EphA2 receptors/cell and R2 were used as a metric for linearity. Partition analysis was performed to evaluate the ability of the assay to classify EphA2+ vs. EphA2− cell lines (using our pre-established mechanistic cutoff of about 3,000 receptors/cell). Partition analysis was also performed to evaluate the ability of the assay to classify EphA2− vs EphA2+ vs EphA2++ cell lines based on the second cutoff of about 17,500 receptors/cell. Intra-assay and inter-assay variability were assessed by computing CV for each cell line, for the slope of the linear regression and for the cutoff of the partition analysis. For analysis of HTMA060915, intra-assay and inter-assay variability were assessed by computing coefficient of variance (CV) for each tumor sample of the TMA. For analysis of cell lines CA111014, qualitative assessed of the staining pattern of EphA2 in the EphA2 overexpressing cell line as it compares to the parental cell line.

Sensitivity and Specificity and Precision Using IHC Staining Quantitative Image Analysis Correlation to Quantitative Flow Cytometry

IHC assay was optimized to enable classification of cell lines into EphA2−, EphA2+, EphA2++. We used our cell line panel and tested several concentrations of primary antibodies keeping all the other parameters of the protocol the same. We tested 0, 1, 2, 6, 10 and 20 μg/ml of primary antibody. We found that the assay was highly tunable and that the error as computed by AUC of ROC was <10% for 1, 2 and 5 μg/ml and was 11 and 14% for 10 and 20 μg/ml. At 20 μg/ml, the upper ranges of the cell lines were saturated. To enable pathologist based detection of the signal we evaluated the strength of the brown staining by eye and found that intensities between 10 and 14 were not visible, and thus we choose a concentration of 2 μg/ml which enables by eye scoring of the staining.

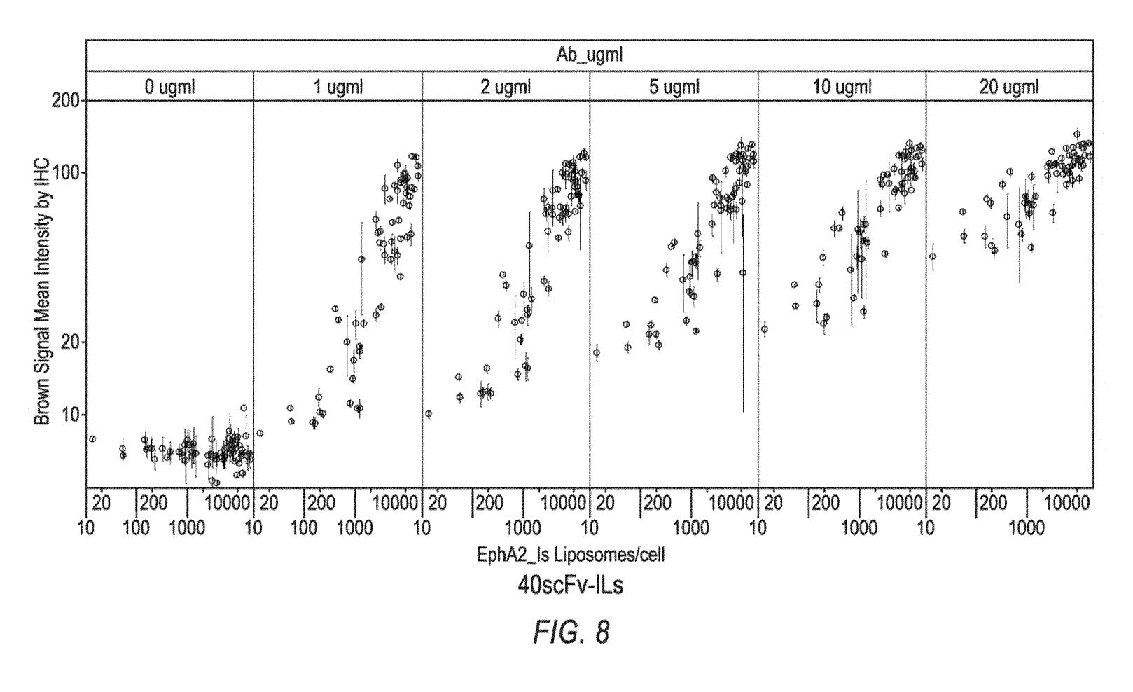

To assess the sensitivity and specificity and precision of the EphA2 IHC assay, the blocks containing 78 cell lines contained 65 unique cell lines with known varying levels of EphA2 expression were sectioned and stained. Correlation and linearity were assessed by analyzing the EPhA2 brown staining vs. receptor per cell (FIG. 9). Partition analysis and ROC analysis was also performed to demonstrate sensitivity and specificity (Table 6). Previous experiments have demonstrated that 3,000 receptors/cell is the sensitivity of this assay and was used to define a cell line as positive or negative for the partition analysis (Table 6). Further clustering of the cell lines allowed the identification of a second in vitro based cutoff of about 17,500 receptors/cell. Taken the two cutoffs together we have grouped the cell lines in three groups EphA2-, EphA2+, EphA2++. Using partition analysis we assessed the ability of the quantitative IHC to classify the cell lines (Table 7).

TABLE 6
Partition analysis for EphA2−/+ cells

| | AUC | FP | FN | TP | TN | Sensitivity | Specificity | PPV | NPV |
|---|---|---|---|---|---|---|---|---|---|
| | ROC | (n) | (n) | (n) | (n) | (%) | (%) | (%) | (%) |
| Run 1.1 | 0.94 | 0 | 2 | 49 | 14 | 96 | 100 | 100 | 88 |
| Run 1.2 | 0.97 | 3 | 0 | 47 | 16 | 100 | 84 | 94 | 100 |
| Run 1.3 | 0.94 | 0 | 2 | 50 | 14 | 96 | 100 | 100 | 88 |
| Run 2.1 | 0.94 | 0 | 2 | 49 | 14 | 96 | 100 | 100 | 88 |
| Run 2.2 | 0.96 | 1 | 1 | 49 | 15 | 98 | 94 | 98 | 94 |
| Run 2.3 | 0.94 | 0 | 2 | 50 | 14 | 96 | 100 | 100 | 88 |
| Run 3.1 | 0.96 | 1 | 1 | 49 | 15 | 98 | 94 | 98 | 94 |
| Run 3.2 | 0.94 | 0 | 2 | 50 | 14 | 96 | 100 | 100 | 88 |
| Run 3.3 | 0.94 | 2 | 1 | 43 | 13 | 98 | 87 | 96 | 93 |
| mean | | | | | | 97 | 95 | 98 | 91 |

IHC assay was able to reproducibly classify the EphA2− and the EphA2+ cell lines with an error<10%. Intra and inter run variability was minimal.

TABLE 7
IHC cutoffs quanitifed through partition analysis enabling classification
of EphA2− cells vs EphA2+ vs EphA2++ cell lines

| | EphA2−/ | EphA2+/ | | | EphA2++ |
|---|---|---|---|---|---|
| | EphA2+ | EphA2++ | EphA2− | EphA2+ | ROC |
| | cutoff | cutoff | ROC AUC | ROC AUC | AUC |
| Run 1.1 | 1.05 | 1.41 | 0.97 | 0.90 | 0.93 |
| Run 1.2 | 1.07 | 1.40 | 0.97 | 0.77 | 0.90 |
| Run 1.3 | 1.03 | 1.42 | 0.98 | 0.86 | 0.90 |
| Run 2.1 | 1.04 | 1.40 | 0.97 | 0.89 | 0.93 |
| Run 2.2 | 1.03 | 1.38 | 0.98 | 0.85 | 0.92 |
| Run 2.3 | 1.02 | 1.38 | 0.98 | 0.86 | 0.90 |
| Run 3.1 | 1.06 | 1.45 | 0.98 | 0.85 | 0.91 |
| Run 3.2 | 1.08 | 1.47 | 0.98 | 0.89 | 0.93 |
| Run 3.3 | 1.06 | 1.43 | 0.97 | 0.78 | 0.90 |

IHC assay was also able to reproducibly classify the EphA2− cell lines with an error ranging from 2 to 3%, EphA2+ cell lines with an error ranging from 22% to 10% and the EphA2++ cell lines with an error ranging from 10% to 7%. Intra and inter precision of the cutoffs show very low variability between runs and within a run.

Intra and inter precision of the quantitative image analysis was also assessed by computing CV at the cell line level. CVs computed in all cell lines showed intra-assay average CV ranging from 1.28%-1.55% with a maximum CV of 6.2%. For inter-assay the average CV was 2.24% and the maximum CV was 23.8% with >95% of the cells lines having CV<20%.

Precision Using Tissue Microarrays

The precision runs using TMA samples consisted of three staining days for three replicate slides and performed by two operators. Below is the description of the run:

Intra- and inter-assay precision will be assessed for reproducibility of staining within the same immunohistochemical staining batch (intra-assay), over separate immunohistochemical staining batches (inter-assay), performed by different operators (inter-operator), and stained on different instruments (inter-instrument). Average brown intensity (units) was extracted from each core including both stroma and tumor tissue. CVs were computed for every core within the run (intra-assay) and between the cores using the average of the three slides within the run (inter-assay) (Table 3). Overall no core reached the % CV maximum permitted level of 20%, and most were below 10%. The median intra-assay CV was 2.9% with 25% percentile 2% and 75% percentile 4.76%. The median inter-assay CV was 2.6% with 25% percentile 1.5% and 75% percentile 3.9%. Intra-assay and inter-assay variability was independent of mean brown intensity (Table 8).

TABLE 8
Intra-assay variability in tumor samples

| | Intra-assay variability | | | |
|---|---|---|---|---|
| | Run1 | Run2 | Run3 | Inter-assay |
| Samples | CV (%) | CV (%) | CV (%) | CV (%) |
| A1 | 0.0 | 2.7 | 3.4 | 4.7 |
| A2 | 2.4 | 3.7 | — | 2.9 |
| A3 | 4.4 | 5.9 | 2.5 | 1.7 |
| A4 | 0.7 | 3.5 | — | 1.5 |
| B1 | 1.3 | 0.4 | — | 0.3 |
| B2 | 0.8 | 1.6 | 0.9 | 0.9 |
| B3 | 2.3 | 4.1 | 2.9 | 2.3 |
| B4 | 3.7 | 5.8 | 4.4 | 2.5 |
| C1 | 2.0 | 4.1 | 1.4 | 2.6 |
| C2 | 2.4 | 2.3 | 2.5 | 2.7 |
| C3 | 1.6 | 12.8 | — | 4.0 |
| C4 | 1.9 | 2.5 | 2.1 | 3.3 |
| D2 | 4.1 | 7.5 | 5.1 | 0.9 |
| D3 | 9.8 | 6.1 | — | 5.8 |
| D4 | 0.9 | — | — | 7.3 |
| D6 | 1.0 | 3.0 | 5.1 | 2.3 |
| E2 | — | — | — | — |
| E3 | 3.5 | 8.2 | 2.6 | 3.6 |
| E4 | 2.9 | 6.2 | 3.9 | 2.9 |
| F2 | 3.3 | 11.4 | 2.9 | 1.0 |
| F3 | 2.6 | 11.6 | 8.2 | 5.5 |

Specificity of EphA2 IHC CDx Tested Using EphA2 Transfected Cell Line.

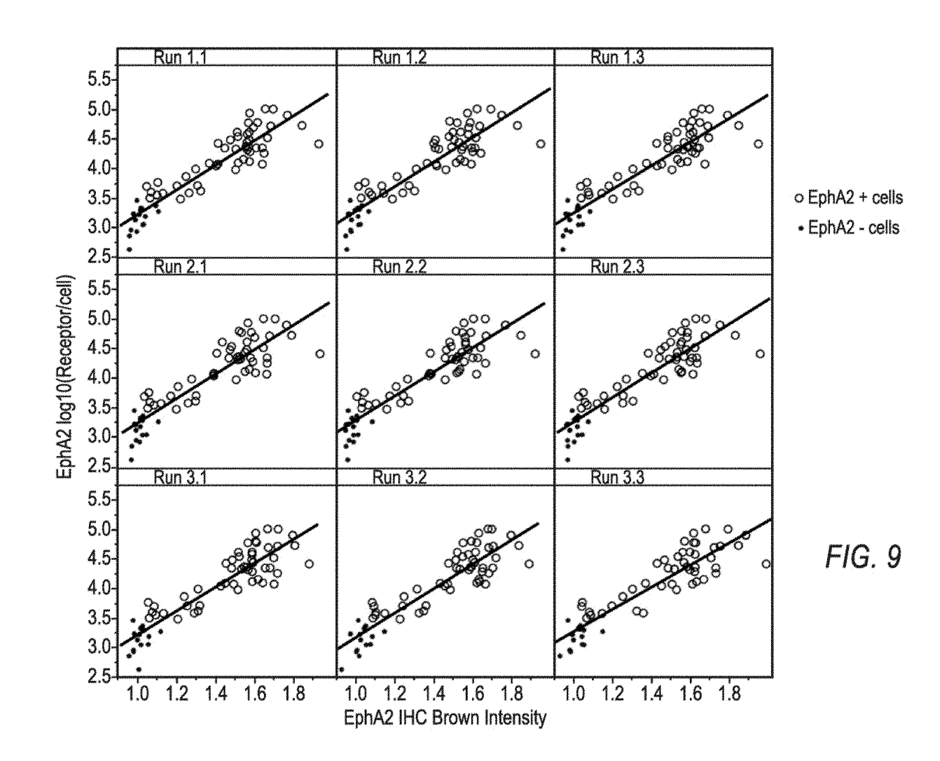

The IGROV-1 cell line was found to have the lowest levels of EphA2 expression by qFACs which was also seen in cell pellets using the EphA2 IHC CDx. We overexpressed EphA2 using a lentiviral construct and confirmed expression by qFACS. Parental IGROV-1 cells have about 1,000 EphA2 receptors/cell while IGROV-1-EPhA2 has about 10000 receptors/cell. Since our mechanistic cutoff is 3,000 receptors/cell, the transfection was able to generate an EphA2+ IGROV cell line. The moderate expression also allows us to qualitatively assess the sensitivity of the assay. Since the analysis is limited to comparing two paired cell lines, we performed qualitative assessment of the staining pattern and have included a snapshot of the cell lines in the report (FIG. 10). EphA2 IHC CDx showed cell membrane staining only in the high EphA2+ cell lines and not in the parental cell line.

EphA2 IHC Scoring Guidance

Control Slide
1. Evaluate the control slide
   a) If quality control criteria are not met, the run is considered failed and the samples EphA2 IHC should not be scored.
   b) Intensity of the staining of the EphA2+ cell line close to the threshold should be evaluated and used as a standard for staining intensity.
   c) An H&E stained section of the tissue sample is recommended for the first evaluation. (The tumor may not be obvious when looking at the sample stained with EphA2 IHC. An H&E stain allows the pathologist to verify the presence of tumor cells).
2. Proceed to scoring the sample. Evaluate and report the percentage positive cancer cells and tumor associated blood vessels. Use decision matrix to identify EphA2 overall score (+ or −) for the sample.

TABLE 9
QC Criteria Definition (Control Slide) for IHC
(Control slide fails if ≥2 QC criteria are not met)

| Sample | Status |
|---|---|
| EphA2− Cell Line | No staining in >90% of cells |
| EphA2+ Cell Line (low) | Dim cell membrane staining in >50% of cells |
| EphA2+ Cell Line (high) | Intense cell membrane staining in >90% of |
| | cells |
| Esophagus Squamous | Gradient of cell membrane staining most |
| Epithelium | intense in the apical side and dim closer to |
| | basal layer. Basal layer is mostly negative. |
| Esophagus muscularis | No or very sparse staining across these tissue |
| mucosa + submucosa + | layers |
| muscularis externa | |

Cancer Cell Scoring (IHC)

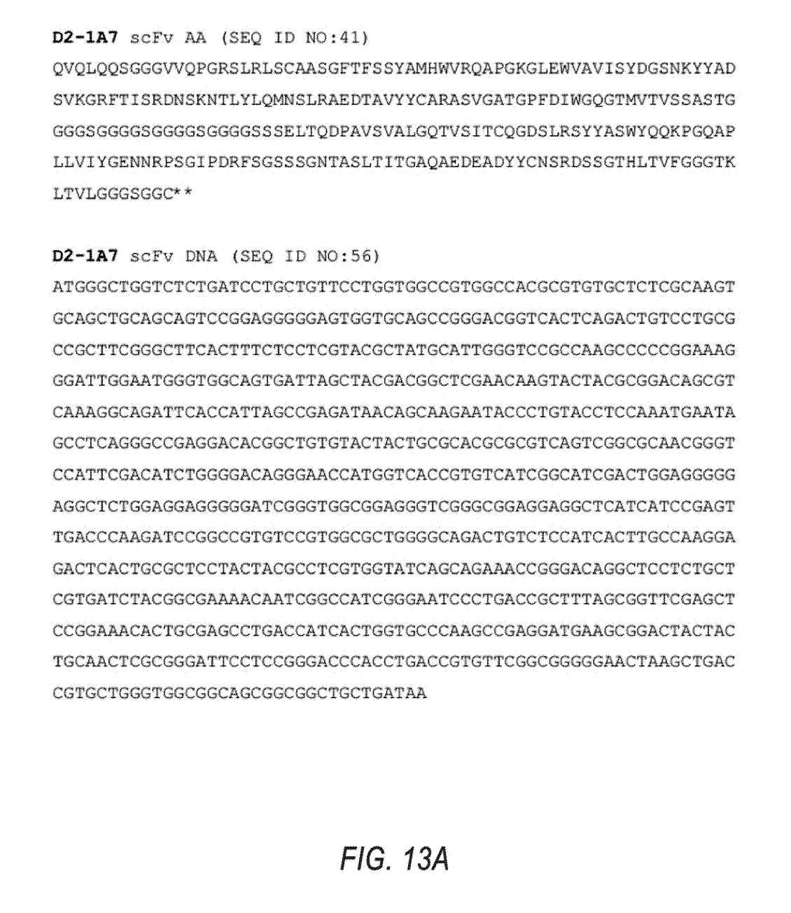

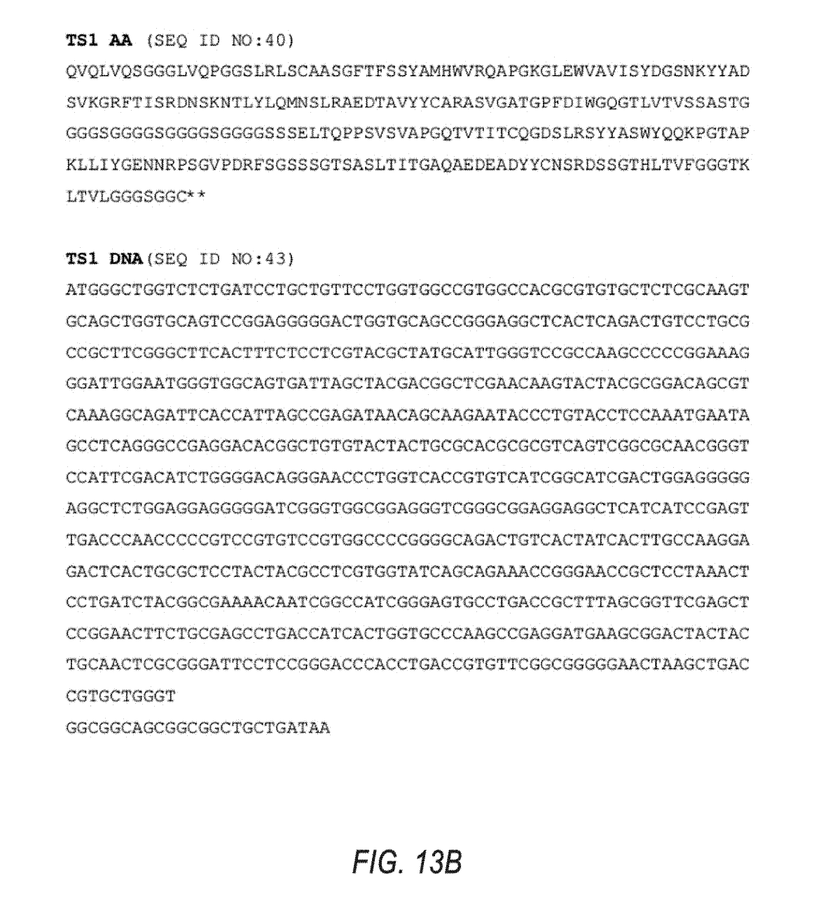

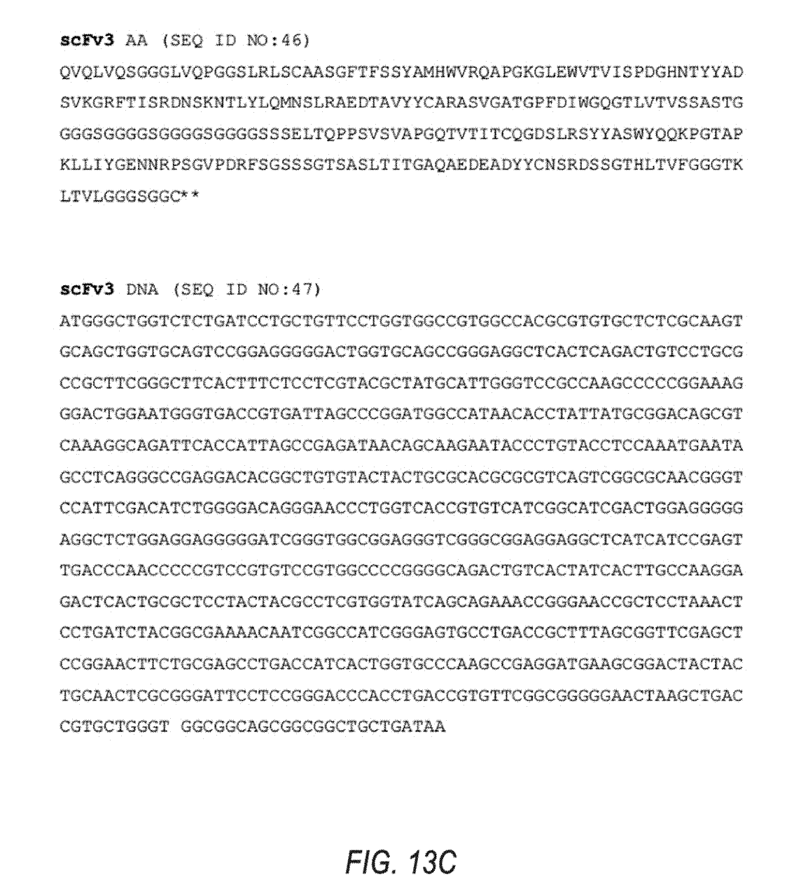

FIG. 13 provides an IHC scoring guide.

Goal is to estimate percentage of positive cancer cells independently of staining intensity. Staining intensity is only referenced to facilitate scoring guidelines.
Evaluate the EphA2 stained sections for estimation of the percentage of tumor cells showing membrane staining at low power first, 4× magnification.
To verify the percentage of stained tumor cells with membrane staining, use 10× magnification. Well-preserved and well-stained areas of the specimen should be used to make a determination of the percent of positive tumor cells

Criteria

If the staining is intense but includes a mixture of cell membrane and cytoplasmic staining patterns or If the staining is very dim confirm the presence of cell membrane location using 20× and 40×.
Cancer cells that have clear staining with average intensity (++ or +++) but mainly cytoplasmic location should be considered EphA2+ and included in the estimation of percent positive cells
Cancer cells that have dim (+) cell membrane staining should be considered EphA2+ and included in the estimation of percentage positive cells
Cancer cells with incomplete membrane staining that only spans a portion of the cell membrane should be included in the estimation of percent positive cells.
Cancer cells that have dim (+) diffuse cytoplasmic staining with no cell membrane pattern should be excluded.
Mixed pattern of intense cell membrane staining with weak and negative cancer cell
20× and/or 40× necessary to confirm cell membrane location in dim (+) cells (black arrow). Red arrow shows negative cells with barely detectable diffuse dim (+) cytoplasmic staining.

Tumor Associated Blood Vessels (TAV) Scoring Guidance

Goal is to estimate percentage of high power fields containing at least one positive TAV independently of staining intensity.

Evaluate the EphA2 sections for estimation of the percentage of positive high power field with at least one EphA2+ TAV at low power first, 10× magnification.

Method
1. Identify 3-4 low power fields (recommended 10×) in or around the tumor areas that include high microvascular density and/or evidence of endothelial staining
2. For each low power field, identify 3 high power fields, 40× magnification for a total high power field number of 12 when possible
3. Evaluate the presence of EphA2 endothelial staining
4. A field is considered positive only if one or more blood vessel is entirely or partially stained.

Report the number of positive and total high power fields

Criteria

Assessment of EphA2+ TAV should be restricted to tissue fragments that include cancer cells. Benign fragments should be excluded from the analysis.

TAV are defined as blood vessels within 2 mm from tumor areas. Blood vessels>2 mm from tumor areas should be excluded from the analysis.

Staining that seems stromal but that is not clearly vascular should be excluded. This applies to non specific staining and myofibroblasts.

Serum staining can be seen and can potentially interfere with endothelial staining assessment. Vessels with weak endothelial staining and serum staining should not be excluded.

EphA2 positive TAV seen at 10× confirmed at higher magnification

Exclude when serum staining artifact hinders endothelial assessment

Exclude when staining not clearly endothelial

### Example 3: EphA2 Targeted Docetaxel Prodrug Nanoliposomes

The EphA2 targeted nano-liposome is preferably a unilamellar lipid bilayer vesicle, approximately 110 nm in diameter, which encapsulates an aqueous space which contains a docetaxel prodrug that converts to docetaxel at a pH present a treatment site.

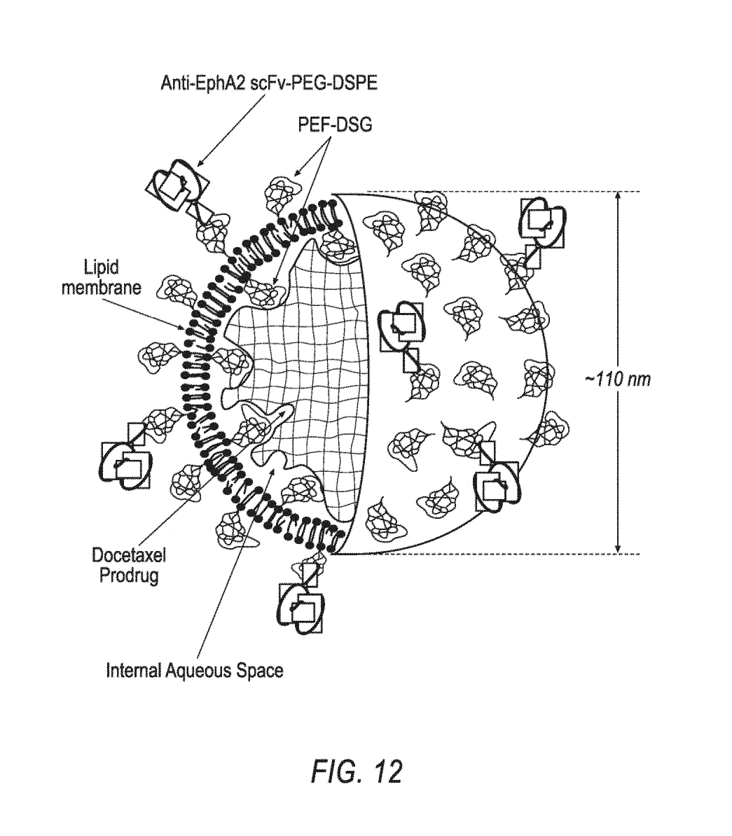

FIG. 12 is a schematic showing the structure of a PEGylated EphA2 targeted liposome encapsulating a docetaxel prodrug. The liposome includes an Ephrin A2 (EphA2) targeted moiety, such as a scFv, bound to the liposome (e.g., through a covalently bound PEG-DSPE moiety). The PEGylated EphA2 targeted liposome encapsulating a docetaxel prodrug can be created by covalently conjugating single chain Fv (scFv) antibody fragments that recognize the EphA2 receptor to pegylated liposomes, containing docetaxel in the form of a prodrug described herein, resulting in an immunoliposomal drug product.

Preferably, the docetaxel prodrug comprises a weak base such as tertiary amine introduced to the 2′ hydroxyl group of docetaxel through ester bond to form a docetaxel prodrug. Preferred 2′-docetaxel prodrugs suitable for loading into a liposome are characterized by comparatively high stability at acidic pH but convert to docetaxel at physiological pH through simple hydrolysis.

The docetaxel prodrug can be stabilized in the liposomal interior during storage and while the intact liposome is in the general circulation, but is hydrolyzed rapidly (e.g., t½=^{˜}10 h) to the active docetaxel upon release from the liposome and entering the environment of the circulating blood.

A docetaxel prodrug can be loaded at mildly acidic pH and entrapped in the acidic interior of liposomes, using an electrochemical gradient where it is stabilized in a non-soluble form.

The docetaxel-generating liposome can comprises a EphA2 targeting moiety. As used herein, unless otherwise indicated, the term “anti-EphA2 scFv” refers to an scFv that immunospecifically binds to EphA2, preferably the ECD of EphA2. An EphA2-specific scFv preferably does not bind to antigens not present in the EphA2 protein. The targeting moiety can be a single chain Fv (“scFv”), a protein that can be covalently bound to a liposome to target the docetaxel-producing liposomes disclosed herein. The scFv can be comprised of a single polypeptide chain in which a VH and a VL are covalently linked to each other, typically via a linker peptide that allows the formation of a functional antigen binding site comprised of VH and VL CDRs. An Ig light or heavy chain variable region is composed of a plurality of “framework” regions (FR) alternating with three hypervariable regions, also called “complementarity determining regions” or “CDRs”.

In certain embodiments, an scFv disclosed herein includes one or any combination of VH FR1, VH FR2, VH FR3, VL FR1, VL FR2, and VL FR3 set forth in Table 10. In one embodiment, the scFv contains the frameworks of the sequences of Table 10 below.

TABLE 10
Exemplary Framework Sequences

| | |
|---|---|
| VH FR1 | QVQLVQSGGGLVQPGGSLRLSCAASGFTFS |
| (SEQ ID NO: 1) | |
| VH FR2 | wvrqapgkglewvT |
| (SEQ ID NO: 2) | |
| VH FR3 | rftisrdnskntlylqmnslraedtavyycar |
| (SEQ ID NO: 3) | |
| VH FR4 | wgqgtlvtvss |
| (SEQ ID NO: 4) | |
| VL FR1 | SsEltqppsvsVapgqTvtiTc |
| (SEQ ID NO: 5) | |
| VL FR2 | wyqqKpgtapklliy |
| (SEQ ID NO: 6) | |
| VL FR3 | gvpdrfsgSSsgtsaslTitgAqaedeadyyc |
| (SEQ ID NO: 7) | |
| VL FR4 | fgggtkltylg |
| (SEQ ID NO: 8) | |

In certain aspects, an scFv disclosed herein is thermostable, e.g., such that the scFv is well-suited for robust and scalable manufacturing. As used herein, a “thermostable” scFv is an scFv having a melting temperature (Tm) of greater than 67° C. or at least about 70° C., e.g., as measured using differential scanning fluorimetry (DSF).

A preferred anti-EphA2 scFv binds to the extracellular domain of EphA2 polypeptide, i.e., the part of the EphA2 protein spanning at least amino acid residues 25 to 534 of the sequence set forth in GenBank Accession No. NP_004422.2 or UniProt Accession No. P29317.

In certain embodiments, an anti-EphA2 scFv disclosed herein includes a VH CDR1, VH CDR2, VH CDR3, VL CDR1, VL CDR2, and VL CDR3 each with a sequence as set forth in Table 11. Note that the VH CDR2 sequence (also referred to as CDRH2) will be any one selected from the 18 different VH CDR2 sequences set forth in Table 11.

TABLE 11
Complementary Determining Regions (CDRs)

| | |
|---|---|
| VH CDR1 (SEQ ID NO: 9) | SYAMH |
| VH CDR2 (SEQ ID NO: 10) | VISPAGNNTYYADSVKG |
| VH CDR2 (SEQ ID NO: 11) | VISPAGRNKYYADSVKG |
| VH CDR2 (SEQ ID NO: 12) | VISPDGHNTYYADSVKG |
| VH CDR2 (SEQ ID NO: 13) | VISPHGRNKYYADSVKG |
| VH CDR2 (SEQ ID NO: 14) | VISRRGDNKYYADSVKG |
| VH CDR2 (SEQ ID NO: 15) | VISNNGHNKYYADSVKG |
| VH CDR2 (SEQ ID NO: 16) | VISPAGPNTYYADSVKG |
| VH CDR2 (SEQ ID NO: 17) | VISPSGHNTYYADSVKG |
| VH CDR2 (SEQ ID NO: 18) | VISPNGHNTYYADSVKG |
| VH CDR2 (SEQ ID NO: 19) | AISPPGHNTYYADSVKG |
| VH CDR2 (SEQ ID NO: 20) | VISPTGANTYYADSVKG |
| VH CDR2 (SEQ ID NO: 21) | VISPHGSNKYYADSVKG |
| VH CDR2 (SEQ ID NO: 22) | VISNNGHNTYYADSVKG |
| VH CDR2 (SEQ ID NO: 23) | VISPAGTNTYYADSVKG |
| VH CDR2 (SEQ ID NO: 24) | VISPPGHNTYYADSVKG |
| VH CDR2 (SEQ ID NO: 25) | VISHDGTNTYYADSVKG |
| VH CDR2 (SEQ ID NO: 26) | VISRHGNNKYYADSVKG |
| VH CDR2 (SEQ ID NO: 27) | VISYDGSNKYYADSVKG |
| VH CDR3 (SEQ ID NO: 28) | asvgatgpfdi |
| VL CDR1 (SEQ ID NO: 29) | qgdslrsyyas |
| VL CDR2 (SEQ ID NO: 30) | gennrps |
| VL CDR3 (SEQ ID NO: 31) | nsrdssgthltv |

In one particular example of a PEGylated EphA2 targeted liposome encapsulating a docetaxel prodrug, the lipid membrane can be composed of N-(hexadecanoyl)-sphing-4-enine-1-phosphocholine (egg sphingomyelin), cholesterol, and 1,2-distearoyl-sn-glycerol, methoxypolyethylene glycol (PEG-DSG). The nanoliposomes can be dispersed in an aqueous buffered solution, such as a sterile pharmaceutical composition formulated for parenteral administration to a human. The PEGylated EphA2 targeted liposome can include the targeting moiety of TS1 (SEQ ID NO:40), D2-1A7 (SEQ ID NO:41) or scFv3 below (SEQ ID NO:46):

TS1 AA
(SEQ ID NO: 40)
QVQLVQSGGGLVQPGGSLRLSCAASGFTFSSYAMHWVRQAPGKGLEWVAV
ISYDGSNKYYADSVKGRFTISRDNSKNTLYLQMNSLRAEDTAVYYCARAS
VGATGPFDIWGQGTLVTVSSASTGGGGSGGGGSGGGGSGGGGSSSELTQP
PSVSVAPGQTVTITCQGDSLRSYYASWYQQKPGTAPKLLIYGENNRPSGV
PDRFSGSSSGTSASLTITGAQAEDEADYYCNSRDSSGTHLTVFGGGTKLT
VLGGGSGGC**
D2-1A7 scFv AA
(SEQ ID NO: 41)
QVQLQQSGGGVVQPGRSLRLSCAASGFTFSSYAMHWVRQAPGKGLEWVAV
ISYDGSNKYYADSVKGRFTISRDNSKNTLYLQMNSLRAEDTAVYYCARAS
VGATGPFDIWGQGTMVTVSSASTGGGGSGGGGSGGGGSGGGGSSSELTQD
PAVSVALGQTVSITCQGDSLRSYYASWYQQKPGQAPLLVIYGENNRPSGI
PDRFSGSSSGNTASLTITGAQAEDEADYYCNSRDSSGTHLTVFGGGTKLT
VLGGGSGGC**
scFv3 AA
(SEQ ID NO: 46)
QVQLVQSGGGLVQPGGSLRLSCAASGFTFSSYAMHWVRQAPGKGLEWVTV
ISPDGHNTYYADSVKGRFTISRDNSKNTLYLQMNSLRAEDTAVYYCARAS
VGATGPFDIWGQGTLVTVSSASTGGGGSGGGGSGGGGSGGGGSSSELTQP
PSVSVAPGQTVTITCQGDSLRSYYASWYQQKPGTAPKLLIYGENNRPSGV
PDRFSGSSSGTSASLTITGAQAEDEADYYCNSRDSSGTHLTVFGGGTKLT
VLGGGSGGC**

An exemplary EphA2 targeted docetaxel-generating nanoliposome composition designated “46scFv-ILs-DTXp3,” a targeted liposome comprising a compound of Formula (I) designated Compound 3 encapsulated in a lipid vesicle formed from egg sphingomyelin, cholesterol and PEG-DSG in a weight ratio of about 4.4:1.6:1, with scFv of SEQ ID NO:46 attached to the lipid vesicle (to provide targeting to EphA2) in a weight ratio of about 1:142 of the total amount of sphingomyelin in the lipid vesicle.

TABLE 12
DTX DERIVATIVES

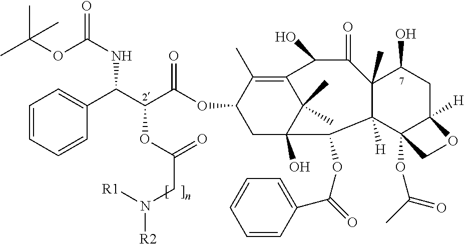

| | | | MW (gram/mol) |
|---|---|---|---|
| Compd. | R₁ | R₂ | HCl Salt (Free Base) |
| 1 | 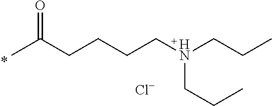 | H | 1027.6 (991.4) |
| 2 | 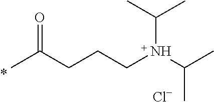 | H | 1013.6 (977.2) |
| 3 | 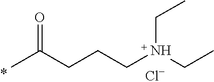 | H | 971.5 (935.1) |
| 4 | 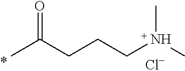 | H | 985.6 (949.1) |
| 5 | 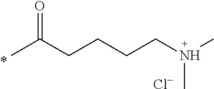 | H | 957.5 (921.1) |
| 6 | 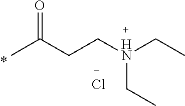 | | 971.5 (935.1) |

Two specific examples of preferred EphA2 targeted docetaxel-generating nanoliposome compositions are 46scFv-ILs-DTXp3 (i.e., the EphA2 targeted docetaxel-generating nanoliposome composition comprising the scFv of SEQ ID NO:46 attached to an immunoliposome encapsulating docetaxel prodrug Compound 3 herein) and 46scFv-ILs-DTXp4. Alternative preferred embodiments can include EphA2 targeted docetaxel-generating immunoliposomes with:
(1) a lipid vesicle formulated with one or more uncharged lipid component (e.g., cholesterol, or other components such as diacylglycerol, acyl(poly ethers) and/or alkylpoly(ethers)), one or more neutral phospholipids (e.g., diacylphosphatidylcholines, sphingomyelins, and/or diacylphosphatidylethanolamines) and a PEGylated lipid component, in any suitable ratio providing desired plasma stability for the targeted immunoliposome;
(2) a docetaxel prodrug suitable for loading into the immunoliposome (e.g., using a trapping agent such as sucrose octasulfate or other sulfonated polyol), such as a compound of Formula (I) herein (preferably a docetaxel prodrug compound of formula (I) where n is 1-4 (preferably 2 or 3) and R¹ and R² are each independently C₁-C₄ alkyl (preferably C₂ or C₃ alkyl); more preferably a docetaxel prodrug compound of formula (I) where n is 2 or 3, and R¹ and R² are each independently C₁-C₃ alkyl; most preferably a docetaxel prodrug compound of formula (I) where n is 2 or 3, and R¹ and R² are each independently ethyl);
(3) lipid vesicle comprising a scFv moiety comprising VH CDR1 of SEQ ID NO:9, VH CDR2 of any one of SEQ ID Nos: 11-27, VH CDR3 of SEQ ID NO: 28, VL CDR1 of SEQ ID NO:29, VL CDR2 of SEQ ID NO:30, and VL CDR3 of SEQ ID NO:31 attached to a PEGylated lipid forming the liposome vesicle (e.g., PEG-DSPE); and
(4) a buffer composition surrounding the EphA2-targeted docetaxel-generating immunoliposome to form a sterile drug product, including a buffer system (e.g., citric acid and sodium citrate), an isotonicity agent (e.g., sodium chloride) and a sterile water vehicle as a diluent (e.g., water for injection).

### Example 4: Nanoliposomal Targeting of Ephrin Receptor A2 (EphA2): Clinical Translation

Ephrin receptor A2 (EphA2) is part of the Ephrin family of cell-cell junction proteins highly overexpressed in several solid tumors, and is associated with poor prognosis. We developed a novel EphA2-targeted docetaxel nanoliposome, leveraging organ specificity through the enhanced permeability and retention effect and cellular specificity through EphA2 targeting. The goal of the study was to develop the diagnostic framework enabling the clinical implementation of EphA2-based exclusion criteria in future MM-310 trials.

We used qFACS and an in vitro assay for liposome (Ls)-cell interaction to identify the minimum number of EphA2 receptors to enable antibody-mediated internalization of Ls. We developed an IHC assay able to differentiate EphA2− vs + cell lines. We characterized EphA2 staining pattern in tumor samples of various indications and developed a scoring algorithm that allows selection of patients in early clinical trials.

While non-targeted Ls do not associate with cells in vitro, there is a strong correlation between EphA2 expression and EphA2-Ls cell association independent of the cell line origin. We used the non-targeted Ls to determine the extent of non-specific binding that can be achieved (^{˜}340 Ls/cell) and used partitioning to determine the minimum number of EphA2 receptors necessary to mediate targeting (^{˜}3000 receptors/cell). We have developed and validated a qIHC assay for EphA2 (precision ^{˜}90%, linearity 0.8 and reproducibility CV<5%). We stained a set of ^{˜}200 tumor samples from various indications. EphA2 was found to be expressed in tumor cells, tumor-associated myofibroblasts, and tumor-associated blood vessels. Using an inclusive cutoff of 10%, EphA2 prevalence was found to range from 50% to 100% in the tumor types evaluated. No significant difference in staining was seen between metastasis and primary tumors in matched samples.

In summary, we developed a diagnostic framework for prospective selection of EphA2+ patients for MM-310 trials based on a mechanistic single cell cut-off and a clinical-grade IHC assay.

TABLE 13

| | | Tumor | Tumor | EphA2 |
|---|---|---|---|---|
| | | associated | associated | Overall |
| | Cancer Cells | myofibroblasts | blood vessels | Score |
| Bladder | 19/20 (95%) | 0/20 (0%) | 16/20 (80%) | 19/20 (95%) |
| Gastric | 18/20 (90%) | 3/20 (15%) | 17/20 (85%) | 20/20 (100%) |
| Head & | 16/19 (84%) | 0/19 (0%) | 9/19 (47%) | 19/19 (100%) |
| Neck | | | | |
| Lung | 24/41 (58%) | 1/41 (2.4%) | 24/41 (58%) | 28/41 (68%) |
| Lung- | 7/9 (78%) | — | — | 7/9 (78%) |
| FNA | | | | |
| Ovarian | 10/18 (55%) | 7/18 (39%) | 17/18 (95%) | 17/18 (95%) |
| Pancreatic | 15/19 (79%) | 0/19 (0%) | 11/19 (58%) | 17/19 (89%) |
| Prostate | 7/23 (27%) | 7/23 (27%) | 9/23 (28%) | 12/23 (52%) |
| TNBC | 6/77 (7%) | 0/77 (0%) | 34/77 (44%) | 37/77 (48%) |

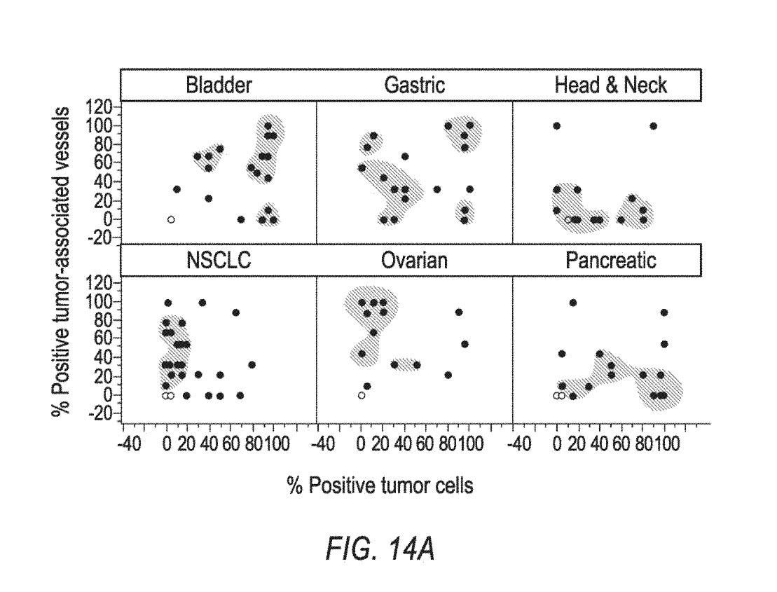

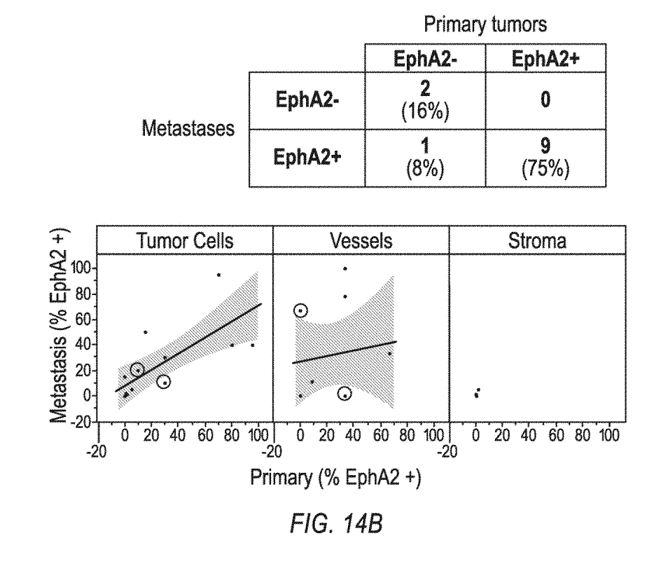

FIG. 14A shows the scatter plot of percent positive tumor cells vs. percent positive tumor associated blood vessels. Several distinct pattern of expression can be seen in the various tested tumor types. In bladder cancer, most of the patients are positive in tumor cells and tumor associated blood vessels with very high percent positivity in both compartments. In gastric cancer the distribution is more even and there's a wide range of expression levels in EphA2+ patients (>=10% in any compartment). Non-small cell lung cancer (NSCLC) and ovarian cancer show a heavier distribution closer to the tumor associated blood vessels, while head & neck and pancreatic have high expression in tumor cells compared to tumor associated blood vessels.

To assess EphA2 expression evolution during disease progression, we evaluated the expression of EphA2 in matched primary/metastasis samples of the same patients. We acquired two sets of samples (1) all indication set of 12 patients (2) a bladder cancer set of 10 patients. EphA2 expression was consistent between primary and metastasis in both sets with a concordance of 91% and 90% in the all indication set and the bladder cancer set respectively.

Primary Tumors

| | |
|---|---|
| EphA2− | EphA2+ |

All indications Set

| | | | |
|---|---|---|---|
| Metastasis | EphA2− | 2 (16%) | 0 |
| | EphA2+ | 1 (8%) | 9 (75%) |

Bladder Cancer

| | | | |
|---|---|---|---|
| Metastasis | EphA2− | 4 (40%) | 0 |
| | EphA2+ | 1 (10%) | 5 (50%) |

In vitro cell binding data was used to identify minimum number of EphA2/cell to allow targeted liposome uptake. Immunohistochemistry assay for EphA2 in formalin-fixed, paraffin embedded tissues was analytically validated and used to survey human tumors from several indications. EphA2 was observed in tumor cells, stroma, and in tumor-associated blood vessels, and was consistently expressed in matched primary tumors and metastases. EphA2 negative patients will be excluded from clinical trials based upon prospective screening results. Retrospective analysis of EphA2 compartment contributions when patient outcome data is available will be used to refine inclusion criteria to best serve patients who would benefit from MM-310.

1. A method of treating an EphA2 positive human cancer in a human patient, the method comprising administering a therapeutically effective amount of a docetaxel prodrug encapsulated in a liposome comprising an EphA2 targeted antibody, to treat the cancer in the human patient.

2. The method of claim 1, wherein the EphA2 positive human cancer comprises cancer cells having at least 3,000 EphA2 per cell.

3. The method of claim 1, wherein the EphA2 targeted scFv antibody comprises an isolated monoclonal antibody that specifically binds an epitope of EphA2, wherein the epitope is specifically bound by a scFv moiety comprising SEQ ID NO:41.

4. The method of claim 1, wherein the docetaxel prodrug comprises a compound of Formula (I).

5. The method of claim 1, wherein the docetaxel prodrug is selected from Compounds 1-6, or a pharmaceutically acceptable salt thereof.

6. The method of claim 5, wherein the docetaxel prodrug is a sucrose octasulfate salt of Compound 3 encapsulated in a liposome.

7. The method of claim 5, wherein the docetaxel prodrug is a sucrose octasulfate salt of Compound 6 encapsulated in a liposome.

8. The method of claim 1, wherein at least 10% of the cells in the tumor overexpress EphA2 and/or at least 10% of the tumor associate blood vessel cells overexpress EphA2.

9. The method of claim 7, wherein the tumor cells and/or tumor associate blood vessel cells comprise cancer cells having an average at least 3,000 EphA2 receptors per cell.

10. A liposome-cell association method for identifying human patients having an EphA2 positive human cancer tumor, the method comprising obtaining a tissue sample of the tumor, and determining that at least 10% of the cells in the tumor overexpress EphA2 and/or at least 10% of the tumor associate blood vessel cells overexpress EphA2.

11-12. (canceled)

13. A liposome-cell association method for identifying human patients having an EphA2 positive human cancer tumor, the method comprising obtaining a tissue sample of the tumor, and determining that at least 10% of the cells in the tumor overexpress EphA2 in the 2+ range (17,500 receptors/cell) and/or at least 10% of the tumor associate blood vessel cells overexpress EphA2.

14. The method of claim 1, wherein the tumor being treated is a solid tumor.

15. The method of claim 13, wherein the solid tumor is chosen from the list of ovarian, pancreatic, breast, lung, and prostate cancer.

16. The method of claim 6, wherein the tumor cells and/or tumor associate blood vessel cells comprise cancer cells having an average at least 3,000 EphA2 receptors per cell.

17. The method of claim 6, wherein the tumor cells have at least an average of 17,500 EphA2 receptors per cell.

18. The method of claim 7, wherein the tumor cells have at least an average of 17,500 EphA2 receptors per cell.


SEQUENCE LISTING (56 sequences)

SEQ ID NO: 1 - protein, 30 aa, Artificial Sequence
  Synthetically Generated Sequence
QVQLVQSGGGLVQPGGSLRLSCAASGFTFS

SEQ ID NO: 2 - protein, 14 aa, Artificial Sequence
  Synthetically Generated Sequence
WVRQAPGKGLEWVT

SEQ ID NO: 3 - protein, 32 aa, Artificial Sequence
  Synthetically Generated Sequence
RFTISRDNSKNTLYLQMNSLRAEDTAVYYCAR

SEQ ID NO: 4 - protein, 11 aa, Artificial Sequence
  Synthetically Generated Sequence
WGQGTLVTVSS

SEQ ID NO: 5 - protein, 22 aa, Artificial Sequence
  Synthetically Generated Sequence
SSELTQPPSVSVAPGQTVTITC

SEQ ID NO: 6 - protein, 15 aa, Artificial Sequence
  Synthetically Generated Sequence
WYQQKPGTAPKLLIY

SEQ ID NO: 7 - protein, 32 aa, Artificial Sequence
  Synthetically Generated Sequence
GVPDRFSGSSSGTSASLTITGAQAEDEADYYC

SEQ ID NO: 8 - protein, 11 aa, Artificial Sequence
  Synthetically Generated Sequence
FGGGTKLTVLG

SEQ ID NO: 9 - protein, 5 aa, Artificial Sequence
  Synthetically Generated Sequence
SYAMH

SEQ ID NO: 10 - protein, 17 aa, Artificial Sequence
  Synthetically Generated Sequence
VISPAGNNTYYADSVKG

SEQ ID NO: 11 - protein, 17 aa, Artificial Sequence
  Synthetically Generated Sequence
VISPAGRNKYYADSVKG

SEQ ID NO: 12 - protein, 17 aa, Artificial Sequence
  Synthetically Generated Sequence
VISPDGHNTYYADSVKG

SEQ ID NO: 13 - protein, 17 aa, Artificial Sequence
  Synthetically Generated Sequence
VISPHGRNKYYADSVKG

SEQ ID NO: 14 - protein, 17 aa, Artificial Sequence
  Synthetically Generated Sequence
VISRRGDNKYYADSVKG

SEQ ID NO: 15 - protein, 17 aa, Artificial Sequence
  Synthetically Generated Sequence
VISNNGHNKYYADSVKG

SEQ ID NO: 16 - protein, 17 aa, Artificial Sequence
  Synthetically Generated Sequence
VISPAGPNTYYADSVKG

SEQ ID NO: 17 - protein, 17 aa, Artificial Sequence
  Synthetically Generated Sequence
VISPSGHNTYYADSVKG

SEQ ID NO: 18 - protein, 17 aa, Artificial Sequence
  Synthetically Generated Sequence
VISPNGHNTYYADSVKG

SEQ ID NO: 19 - protein, 17 aa, Artificial Sequence
  Synthetically Generated Sequence
AISPPGHNTYYADSVKG

SEQ ID NO: 20 - protein, 17 aa, Artificial Sequence
  Synthetically Generated Sequence
VISPTGANTYYADSVKG

SEQ ID NO: 21 - protein, 17 aa, Artificial Sequence
  Synthetically Generated Sequence
VISPHGSNKYYADSVKG

SEQ ID NO: 22 - protein, 17 aa, Artificial Sequence
  Synthetically Generated Sequence
VISNNGHNTYYADSVKG

SEQ ID NO: 23 - protein, 17 aa, Artificial Sequence
  Synthetically Generated Sequence
VISPAGTNTYYADSVKG

SEQ ID NO: 24 - protein, 17 aa, Artificial Sequence
  Synthetically Generated Sequence
VISPPGHNTYYADSVKG

SEQ ID NO: 25 - protein, 17 aa, Artificial Sequence
  Synthetically Generated Sequence
VISHDGTNTYYADSVKG

SEQ ID NO: 26 - protein, 17 aa, Artificial Sequence
  Synthetically Generated Sequence
VISRHGNNKYYADSVKG

SEQ ID NO: 27 - protein, 18 aa, Artificial Sequence
  Synthetically Generated Sequence
VISYDGSNKYYADSVKGG

SEQ ID NO: 28 - protein, 11 aa, Artificial Sequence
  Synthetically Generated Sequence
ASVGATGPFDI

SEQ ID NO: 29 - protein, 11 aa, Artificial Sequence
  Synthetically Generated Sequence
QGDSLRSYYAS

SEQ ID NO: 30 - protein, 7 aa, Artificial Sequence
  Synthetically Generated Sequence
GENNRPS

SEQ ID NO: 31 - protein, 12 aa, Artificial Sequence
  Synthetically Generated Sequence
NSRDSSGTHLTV

SEQ ID NO: 32 - protein, 23 aa, Artificial Sequence
  Synthetically Generated Sequence
ASTGGGGSGGGGSGGGGSGGGGS

SEQ ID NO: 33 - protein, 20 aa, Artificial Sequence
  Synthetically Generated Sequence
GGGGSGGGGSGGGGSGGGGS

SEQ ID NO: 34 - protein, 15 aa, Artificial Sequence
  Synthetically Generated Sequence
GGGGSGGGGSGGGGS

SEQ ID NO: 35 - protein, 23 aa, Artificial Sequence
  Synthetically Generated Sequence
ASTGGGGAGGGGAGGGGAGGGGA

SEQ ID NO: 36 - protein, 20 aa, Artificial Sequence
  Synthetically Generated Sequence
GGGGAGGGGAGGGGAGGGGA

SEQ ID NO: 37 - protein, 24 aa, Artificial Sequence
  Synthetically Generated Sequence
TPSHNSHQVPSAGGPTANSGTSGS

SEQ ID NO: 38 - protein, 18 aa, Artificial Sequence
  Synthetically Generated Sequence
GGSSRSSSSGGGGSGGGG

SEQ ID NO: 39 - protein, 457 aa, Artificial Sequence
  Synthetically Generated Sequence
EVQLLESGGGLVQPGGSLRLSCAASGFTFSHYMMAWVRQAPGKGLEWVSRIGPSGGPTHYADSVKGRFTISRDNSKNTLYLQMNSLRAEDTAVYYCAGYDSGYDYVAVAGPAEYFQHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 40 - protein, 259 aa, Artificial Sequence
  Synthetically Generated Sequence
QVQLVQSGGGLVQPGGSLRLSCAASGFTFSSYAMHWVRQAPGKGLEWVAVISYDGSNKYYADSVKGRFTISRDNSKNTLYLQMNSLRAEDTAVYYCARASVGATGPFDIWGQGTLVTVSSASTGGGGSGGGGSGGGGSGGGGSSSELTQPPSVSVAPGQTVTITCQGDSLRSYYASWYQQKPGTAPKLLIYGENNRPSGVPDRFSGSSSGTSASLTITGAQAEDEADYYCNSRDSSGTHLTVFGGGTKLTVLGGGSGGC

SEQ ID NO: 41 - protein, 259 aa, Artificial Sequence
  Synthetically Generated Sequence
QVQLQQSGGGVVQPGRSLRLSCAASGFTFSSYAMHWVRQAPGKGLEWVAVISYDGSNKYYADSVKGRFTISRDNSKNTLYLQMNSLRAEDTAVYYCARASVGATGPFDIWGQGTMVTVSSASTGGGGSGGGGSGGGGSGGGGSSSELTQDPAVSVALGQTVSITCQGDSLRSYYASWYQQKPGQAPLLVIYGENNRPSGIPDRFSGSSSGNTASLTITGAQAEDEADYYCNSRDSSGTHLTVFGGGTKLTVLGGGSGGC

SEQ ID NO: 42 - protein, 214 aa, Artificial Sequence
  Synthetically Generated Sequence
DIQMTQSPSSLSASVGDRVTITCRASQSISTWLAWYQQKPGKAPKLLIYKASNLHTGVPSRFSGSGSGTEFSLTISGLQPDDFATYYCQQYNSYSRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - DNA, 840 bp, Artificial Sequence
  Synthetically Generated Sequence
atgggctggtctctgatcctgctgttcctggtggccgtggccacgcgtgtgctctcgcaagtgcagctggtgcagtccggagggggactggtgcagccgggaggctcactcagactgtcctgcgccgcttcgggcttcactttctcctcgtacgctatgcattgggtccgccaagcccccggaaagggattggaatgggtggcagtgattagctacgacggctcgaacaagtactacgcggacagcgtcaaaggcagattcaccattagccgagataacagcaagaataccctgtacctccaaatgaatagcctcagggccgaggacacggctgtgtactactgcgcacgcgcgtcagtcggcgcaacgggtccattcgacatctggggacagggaaccctggtcaccgtgtcatcggcatcgactggagggggaggctctggaggagggggatcgggtggcggagggtcgggcggaggaggctcatcatccgagttgacccaacccccgtccgtgtccgtggccccggggcagactgtcactatcacttgccaaggagactcactgcgctcctactacgcctcgtggtatcagcagaaaccgggaaccgctcctaaactcctgatctacggcgaaaacaatcggccatcgggagtgcctgaccgctttagcggttcgagctccggaacttctgcgagcctgaccatcactggtgcccaagccgaggatgaagcggactactactgcaactcgcgggattcctccgggacccacctgaccgtgttcggcgggggaactaagctgaccgtgctgggtggcggcagcggcggctgctgataa

SEQ ID NO: 44 - protein, 259 aa, Artificial Sequence
  Synthetically Generated Sequence
QVQLVQSGGGLVQPGGSLRLSCAASGFTFSSYAMHWVRQAPGKGLEWVSVISPAGRNKYYADSVKGRFTISRDNSKNTLYLQMNSLRAEDTAVYYCARASVGATGPFDIWGQGTLVTVSSASTGGGGSGGGGSGGGGSGGGGSSSELTQPPSVSVAPGQTVTITCQGDSLRSYYASWYQQKPGTAPKLLIYGENNRPSGVPDRFSGSSSGTSASLTITGAQAEDEADYYCNSRDSSGTHLTVFGGGTKLTVLGGGSGGC

SEQ ID NO: 45 - DNA, 840 bp, Artificial Sequence
  Synthetically Generated Sequence
atgggctggtctctgatcctgctgttcctggtggccgtggccacgcgtgtgctctcgcaagtgcagctggtgcagtccggagggggactggtgcagccgggaggctcactcagactgtcctgcgccgcttcgggcttcactttctcctcgtacgctatgcattgggtccgccaagcccccggaaagggactggaatgggtgagcgtgattagcccggcgggccgcaacaaatattatgcggacagcgtcaaaggcagattcaccattagccgagataacagcaagaataccctgtacctccaaatgaatagcctcagggccgaggacacggctgtgtactactgcgcacgcgcgtcagtcggcgcaacgggtccattcgacatctggggacagggaaccctggtcaccgtgtcatcggcatcgactggagggggaggctctggaggagggggatcgggtggcggagggtcgggcggaggaggctcatcatccgagttgacccaacccccgtccgtgtccgtggccccggggcagactgtcactatcacttgccaaggagactcactgcgctcctactacgcctcgtggtatcagcagaaaccgggaaccgctcctaaactcctgatctacggcgaaaacaatcggccatcgggagtgcctgaccgctttagcggttcgagctccggaacttctgcgagcctgaccatcactggtgcccaagccgaggatgaagcggactactactgcaactcgcgggattcctccgggacccacctgaccgtgttcggcgggggaactaagctgaccgtgctgggtggcggcagcggcggctgctgataa

SEQ ID NO: 46 - protein, 259 aa, Artificial Sequence
  Synthetically Generated Sequence
QVQLVQSGGGLVQPGGSLRLSCAASGFTFSSYAMHWVRQAPGKGLEWVTVISPDGHNTYYADSVKGRFTISRDNSKNTLYLQMNSLRAEDTAVYYCARASVGATGPFDIWGQGTLVTVSSASTGGGGSGGGGSGGGGSGGGGSSSELTQPPSVSVAPGQTVTITCQGDSLRSYYASWYQQKPGTAPKLLIYGENNRPSGVPDRFSGSSSGTSASLTITGAQAEDEADYYCNSRDSSGTHLTVFGGGTKLTVLGGGSGGC

SEQ ID NO: 47 - DNA, 861 bp, Artificial Sequence
  Synthetically Generated Sequence
atgggctggtctctgatcctgctgttcctggtggccgtggccacgcgtgtgctctcgcaagtgcagctggtgcagtccggagggggactggtgcagccgggaggctcactcagactgtcctgcgccgcttcgggcttcactttctcctcgtacgctatgcattgggtccgccaagcccccggaaagggactggaatgggtgaccgtgattagcccggatggccataacacctattatgcggacagcgtcaaaggcagattcaccattagccgagataacagcaagaataccctgtacctccaaatgaatagcctcagggccgaggacacggctgtgtactactgcgcacgcgcgtcagtcggcgcaacgggtccattcgacatctggggacagggaaccctggtcaccgtgtcatcggcatcgactggagggggaggctctggaggagggggatcgggtggcggagggtcgggcggaggaggctcatcatccgagttgacccaacccccgtccgtgtccgtggccccggggcagactgtcactatcacttgccaaggagactcactgcgctcctactacgcctcgtggtatcagcagaaaccgggaaccgctcctaaactcctgatctacggcgaaaacaatcggccatcgggagtgcctgaccgctttagcggttcgagctccggaacttctgcgagcctgaccatcactggtgcccaagccgaggatgaagcggactactactgcaactcgcgggattcctccgggacccacctgaccgtgttcggcgggggaactaagctgaccgtgctgggtcgtacggtggcggcgcccagtcaccatcatcatcaccactgataa

SEQ ID NO: 48 - protein, 259 aa, Artificial Sequence
  Synthetically Generated Sequence
QVQLVQSGGGLVQPGGSLRLSCAASGFTFSSYAMHWVRQAPGKGLEWVTVISPSGHNTYYADSVKGRFTISRDNSKNTLYLQMNSLRAEDTAVYYCARASVGATGPFDIWGQGTLVTVSSASTGGGGSGGGGSGGGGSGGGGSSSELTQPPSVSVAPGQTVTITCQGDSLRSYYASWYQQKPGTAPKLLIYGENNRPSGVPDRFSGSSSGTSASLTITGAQAEDEADYYCNSRDSSGTHLTVFGGGTKLTVLGGGSGGC

SEQ ID NO: 49 - DNA, 840 bp, Artificial Sequence
  Snythetically Generated Sequence
atgggctggtctctgatcctgctgttcctggtggccgtggccacgcgtgtgctctcgcaagtgcagctggtgcagtccggagggggactggtgcagccgggaggctcactcagactgtcctgcgccgcttcgggcttcactttctcctcgtacgctatgcattgggtccgccaagcccccggaaagggactggaatgggtgaccgtgattagcccgagcggccataacacctattatgcggacagcgtcaaaggcagattcaccattagccgagataacagcaagaataccctgtacctccaaatgaatagcctcagggccgaggacacggctgtgtactactgcgcacgcgcgtcagtcggcgcaacgggtccattcgacatctggggacagggaaccctggtcaccgtgtcatcggcatcgactggagggggaggctctggaggagggggatcgggtggcggagggtcgggcggaggaggctcatcatccgagttgacccaacccccgtccgtgtccgtggccccggggcagactgtcactatcacttgccaaggagactcactgcgctcctactacgcctcgtggtatcagcagaaaccgggaaccgctcctaaactcctgatctacggcgaaaacaatcggccatcgggagtgcctgaccgctttagcggttcgagctccggaacttctgcgagcctgaccatcactggtgcccaagccgaggatgaagcggactactactgcaactcgcgggattcctccgggacccacctgaccgtgttcggcgggggaactaagctgaccgtgctgggtggcggcagcggcggctgctgataa

SEQ ID NO: 50 - protein, 259 aa, Artificial Sequence
  Synthetically Generated Sequence
QVQLVQSGGGLVQPGGSLRLSCAASGFTFSSYAMHWVRQAPGKGLEWVTVISPNGHNTYYADSVKGRFTISRDNSKNTLYLQMNSLRAEDTAVYYCARASVGATGPFDIWGQGTLVTVSSASTGGGGSGGGGSGGGGSGGGGSSSELTQPPSVSVAPGQTVTITCQGDSLRSYYASWYQQKPGTAPKLLIYGENNRPSGVPDRFSGSSSGTSASLTITGAQAEDEADYYCNSRDSSGTHLTVFGGGTKLTVLGGGSGGC

SEQ ID NO: 51 - DNA, 840 bp, Artificial Sequence
  Synthetically Generated Sequence
atgggctggtctctgatcctgctgttcctggtggccgtggccacgcgtgtgctctcgcaagtgcagctggtgcagtccggagggggactggtgcagccgggaggctcactcagactgtcctgcgccgcttcgggcttcactttctcctcgtacgctatgcattgggtccgccaagcccccggaaagggactggaatgggtgaccgtgattagcccgaacggccataacacctattatgcggacagcgtcaaaggcagattcaccattagccgagataacagcaagaataccctgtacctccaaatgaatagcctcagggccgaggacacggctgtgtactactgcgcacgcgcgtcagtcggcgcaacgggtccattcgacatctggggacagggaaccctggtcaccgtgtcatcggcatcgactggagggggaggctctggaggagggggatcgggtggcggagggtcgggcggaggaggctcatcatccgagttgacccaacccccgtccgtgtccgtggccccggggcagactgtcactatcacttgccaaggagactcactgcgctcctactacgcctcgtggtatcagcagaaaccgggaaccgctcctaaactcctgatctacggcgaaaacaatcggccatcgggagtgcctgaccgctttagcggttcgagctccggaacttctgcgagcctgaccatcactggtgcccaagccgaggatgaagcggactactactgcaactcgcgggattcctccgggacccacctgaccgtgttcggcgggggaactaagctgaccgtgctgggtggcggcagcggcggctgctgataa

SEQ ID NO: 52 - protein, 259 aa, Artificial Sequence
  Synthetically Generated Sequence
QVQLVQSGGGLVQPGGSLRLSCAASGFTFSSYAMHWVRQAPGKGLEWVSAISPPGHNTYYADSVKGRFTISRDNSKNTLYLQMNSLRAEDTAVYYCARASVGATGPFDIWGQGTLVTVSSASTGGGGSGGGGSGGGGSGGGGSSSELTQPPSVSVAPGQTVTITCQGDSLRSYYASWYQQKPGTAPKLLIYGENNRPSGVPDRFSGSSSGTSASLTITGAQAEDEADYYCNSRDSSGTHLTVFGGGTKLTVLGGGSGGC

SEQ ID NO: 53 - DNA, 816 bp, Artificial Sequence
  Synthetically Generated Sequence
atgggctggtctctgatcctgctgttcctggtggccgtggccacgcgtgtgctctcgcaagtgcagctggtgcagtccggagggggactggtgcagccgggaggctcactcagactgtcctgcgccgcttcgggcttcactttctcctcgtacgctatgcattgggtccgccaagcccccggaaagggactggaatgggtgagcgcgattagcccgccgggccataacacctattatgcggacagcgtcaaaggcagattcaccattagccgagataacagcaagaataccctgtacctccaaatgaatagcctcagggccgaggacacggctgtgtactactgcgcacgcgcgtcagtcggcgcaacgggtccattcgacatctggggacagggaaccctggtcaccgtgtcatcggcatcgactggagggggaggctctggaggagggggatcgggtggcggagggtcgggcggaggaggctcatcatccgagttgacccaacccccgtccgtgtccgtggccccggggcagactgtcactatcacttgccaaggagactcactgcgctcctactacgcctcgtggtatcagcagaaaccgggaaccgctcctaaactcctgatctacggcgaaaacaatcggccatcgggagtgcctgaccgctttagcggttcgagctccggaacttctgcgagcctgaccatcactggtgcccaagccgaggatgaagcggactactactgcaactcgcgggattcctccgggacccacctgaccgtgttcggcgggggaactaagctgaccgtgctgggt

SEQ ID NO: 54 - protein, 259 aa, Artificial Sequence
  Synthetically Generated Sequence
QVQLVQSGGGLVQPGGSLRLSCAASGFTFSSYAMHWVRQAPGKGLEWVTVISPTGANTYYADSVKGRFTISRDNSKNTLYLQMNSLRAEDTAVYYCARASVGATGPFDIWGQGTLVTVSSASTGGGGSGGGGSGGGGSGGGGSSSELTQPPSVSVAPGQTVTITCQGDSLRSYYASWYQQKPGTAPKLLIYGENNRPSGVPDRFSGSSSGTSASLTITGAQAEDEADYYCNSRDSSGTHLTVFGGGTKLTVLGGGSGGC

SEQ ID NO: 55 - DNA, 840 bp, Artificial Sequence
  Synthetically Generated Sequence
atgggctggtctctgatcctgctgttcctggtggccgtggccacgcgtgtgctctcgcaagtgcagctggtgcagtccggagggggactggtgcagccgggaggctcactcagactgtcctgcgccgcttcgggcttcactttctcctcgtacgctatgcattgggtccgccaagcccccggaaagggactggaatgggtgaccgtgattagcccgaccggcgcgaacacctattatgcggacagcgtcaaaggcagattcaccattagccgagataacagcaagaataccctgtacctccaaatgaatagcctcagggccgaggacacggctgtgtactactgcgcacgcgcgtcagtcggcgcaacgggtccattcgacatctggggacagggaaccctggtcaccgtgtcatcggcatcgactggagggggaggctctggaggagggggatcgggtggcggagggtcgggcggaggaggctcatcatccgagttgacccaacccccgtccgtgtccgtggccccggggcagactgtcactatcacttgccaaggagactcactgcgctcctactacgcctcgtggtatcagcagaaaccgggaaccgctcctaaactcctgatctacggcgaaaacaatcggccatcgggagtgcctgaccgctttagcggttcgagctccggaacttctgcgagcctgaccatcactggtgcccaagccgaggatgaagcggactactactgcaactcgcgggattcctccgggacccacctgaccgtgttcggcgggggaactaagctgaccgtgctgggtggcggcagcggcggctgctgataa

SEQ ID NO: 56 - DNA, 840 bp, Artificial Sequence
  Synthetically Generated Sequence
atgggctggtctctgatcctgctgttcctggtggccgtggccacgcgtgtgctctcgcaagtgcagctgcagcagtccggagggggagtggtgcagccgggacggtcactcagactgtcctgcgccgcttcgggcttcactttctcctcgtacgctatgcattgggtccgccaagcccccggaaagggattggaatgggtggcagtgattagctacgacggctcgaacaagtactacgcggacagcgtcaaaggcagattcaccattagccgagataacagcaagaataccctgtacctccaaatgaatagcctcagggccgaggacacggctgtgtactactgcgcacgcgcgtcagtcggcgcaacgggtccattcgacatctggggacagggaaccatggtcaccgtgtcatcggcatcgactggagggggaggctctggaggagggggatcgggtggcggagggtcgggcggaggaggctcatcatccgagttgacccaagatccggccgtgtccgtggcgctggggcagactgtctccatcacttgccaaggagactcactgcgctcctactacgcctcgtggtatcagcagaaaccgggacaggctcctctgctcgtgatctacggcgaaaacaatcggccatcgggaatccctgaccgctttagcggttcgagctccggaaacactgcgagcctgaccatcactggtgcccaagccgaggatgaagcggactactactgcaactcgcgggattcctccgggacccacctgaccgtgttcggcgggggaactaagctgaccgtgctgggtggcggcagcggcggctgctgataa